SEQUENCE LISTING (19 sequences)

SEQ ID NO: 1 - protein, 155 aa, Homo sapiens [sequence omitted]

SEQ ID NO: 2 - protein, 196 aa, Homo sapiens
MGGRGRGRAPERVGGRGRGRGTAAPRAAPAARGSRPGPAGTMAAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 3 - protein, 210 aa, Homo sapiens
MGDRGRGRALPGGRLGGRGRGRAPERVGGRGRGRGTAAPRAAPAARGSRPGPAGTMAAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 4 - protein, 288 aa, Homo sapiens
MVGVGGGDVEDVTPRPGGCQISGRGARGCNGIPGAAAWEAALPRRRPRRHPSVNPRSRAAGSPRTRGRRTEERPSGSRLGDRGRGRALPGGRLGGRGRGRAPERVGGRGRGRGTAAPRAAPAARGSRPGPAGTMAAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 5 - protein, 234 aa, Homo sapiens
MPCISGKPWWESRFSWRAPNGGSCEGLCRQVPVPQWLPKLVCGKCGWTGPGHSARAWGIHGVLLPPAGSEDSLDTSLRAHGVHATKHVPEKRPLPIRRKRSIEEAVPAVCKTRTVIYEIPRSQVDPTSANFLIWPPCVEVKRCTGCCNTSSVKCQPSRVHHRSVKVAKVEYVRKKPKLKEVQVRLEEHLECACATTSLNPDYREEDTGEWLPSSASVLENRSSEPCFWGVRWPP

SEQ ID NO: 6 - protein, 241 aa, Homo sapiens
MNRCWALFLSLCCYLRLVSAEGDPIPEELYEMLSDHSIRSFDDLQRLLHGDPGEEDGAELDLNMTRSHSGGELESLARGRRSLGSLTIAEPAMIAECKTRTEVFEISRRLIDRTNANFLVWPPCVEVQRCSGCCNNRNVQCRPTQVQLRPVQVRKIEIVRKKPIFKKATVTLEDHLACKCETVAAARPVTRSPGGSQEQRAKTPQTRVTIRTVRVRRPPKGKHRKFKHTHDKTALKETLGA

SEQ ID NO: 7 - protein, 345 aa, Homo sapiens
MSLFGLLLLTSALAGQRQGTQAESNLSSKFQFSSNKEQNGVQDPQHERIITVSTNGSIHSPRFPHTYPRNTVLVWRLVAVEENVWIQLTFDERFGLEDPEDDICKYDFVEVEEPSDGTILGRWCGSGTVPGKQISKGNQIRIRFVSDEYFPSEPGFCIHYNIVMPQFTEAVSPSVLPPSALPLDLLNNAITAFSTLEDLIRYLEPERWQLDLEDLYRPTWQLLGKAFVFGRKSRVVDLNLLTEEVRLYSCTPRNFSVSIREELKRTDTIFWPGCLLVKRCGGNCACCLHNCNECQCVPSKVTKKYHEVLQLRPKTGVRGLHKSLTDVALEHHEECDCVCRGSTGG

SEQ ID NO: 8 - protein, 370 aa, Homo sapiens
MHRLIFVYTLICANFCSCRDTSATPQSASIKALRNANLRRDESNHLTDLYRRDETIQVKGNGYVQSPRFPNSYPRNLLLTWRLHSQENTRIQLVFDNQFGLEEAENDICRYDFVEVEDISETSTIIRGRWCGHKEVPPRIKSRTNQIKITFKSDDYFVAKPGFKIYYSLLEDFQPAAASETNWESVTSSISGVSYNSPSVTDPTLIADALDKKIAEFDTVEDLLKYFNPESWQEDLENMYLDTPRYRGRSYHDRKSKVDLDRLNDDAKRYSCTPRNYSVNIREELKLANVVFFPRCLLVQRCGGNCGCGTVNWRSCTCNSGKTVKKYHEVLQFEPGHIKRRGRAKTMALVDIQLDHHERCDCICSSRPPR

SEQ ID NO: 9 - protein, 195 aa, Homo sapiens
MGKISSLPTQLFKCCFCDFLKVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKYQPPSTNKNTKSQRRKGWPKTHPGGEQKEGTEASLQIRGKKKEQRREIGSRNAECRGKKGK

SEQ ID NO: 10 - protein, 153 aa, Homo sapiens
MGKISSLPTQLFKCCFCDFLKVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKEVHLKNASRGSAGNKNYRM

SEQ ID NO: 11 - protein, 137 aa, Homo sapiens
MITPTVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKEVHLKNASRGSAGNKNYRM

SEQ ID NO: 12 - protein, 180 aa, Homo sapiens
MGIPMGKSMLVLLTFLAFASCCIAAYRPSETLCGGELVDTLQFVCGDRGFYFSRPASRVSRRSRGIVEECCFRSCDLALLETYCATPAKSERDVSTPPTVLPDNFPRYPVGKFFQYDTWKQSTQRLRRGLPALLRARRGHVLAKELEAFREAKRHRPLIALPTQDPAHGGAPPEMASNRK

SEQ ID NO: 13 - protein, 183 aa, Homo sapiens
MGIPMGKSMLVLLTFLAFASCCIAAYRPSETLCGGELVDTLQFVCGDRGFYFRLPGRPASRVSRRSRGIVEECCFRSCDLALLETYCATPAKSERDVSTPPTVLPDNFPRYPVGKFFQYDTWKQSTQRLRRGLPALLRARRGHVLAKELEAFREAKRHRPLIALPTQDPAHGGAPPEMASNRK

SEQ ID NO: 14 - protein, 147 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKCDKPRR

SEQ ID NO: 15 - protein, 171 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVCDKPRR

SEQ ID NO: 16 - protein, 191 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 17 - protein, 191 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRSLTRKD

SEQ ID NO: 18 - protein, 215 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVPCGPCSERRKHLFVQDPQTCKCSCKNTDSRCKARQLELNERTCRCDKPRR

SEQ ID NO: 19 - protein, 232 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

(19) United States
(12) Patent Application Publication
(10) Pub. No.: US 2018/0085434 A1
(43) Pub. Date: Mar. 29, 2018

(54) COMPOSITION

(71) Applicant: Cell Therapy Limited, Cardiff, South Wales (GB)

(72) Inventors: Martin John Evans, Cardiff, South Wales (GB); Ajan Reginald, Cardiff, South Wales (GB); Sabena Sultan, Cardiff, South Wales (GB)

(21) Appl. No.: 15/559,411

(22) PCT Filed: Mar. 17, 2016
(86) PCT No.: PCT/GB2016/050744

§ 371 Date: Sep. 18, 2017

(30) Foreign Application Priority Data
Mar. 19, 2015 (GB) 1504665.9

(51) Int. Cl. A61K 38/18; A61K 9/06; A61K 47/38; A61K 35/19; C12N 5/078; C12N 5/0735; C12N 5/074; A61P 15/02; A61K 8/64; A61Q 7/00; A61P 21/00

(52) U.S. Cl. CPC A61K 38/1858; A61K 38/1825; A61K 38/1891; A61K 9/06; A61K 47/38; A61K 35/19; A61K 2035/124; C12N 5/0606; C12N 5/0607; A61P 15/02; A61K 8/64; A61Q 7/00; A61P 21/00; C12N 5/0634

(57) ABSTRACT

The invention concerns a composition comprising basic fibroblast growth factor and platelet derived growth factor, and its use to treat tissue damage or inhibit senescence, or promote hair growth.

### FIELD OF THE INVENTION

The invention concerns a composition comprising basic fibroblast growth factor and platelet derived growth factor, and its use to treat tissue damage, inhibit senescence, or promote hair growth.

### BACKGROUND OF THE INVENTION

A wide variety of injuries and pathological processes can result in tissue. Repair of the damaged tissue is dependent upon tissue regeneration, the growth of new tissue from healthy neighbouring tissue to replace the damaged tissue. Tissue regeneration is tightly controlled at the physiological, cellular and molecular levels, and requires the action and balance of a number of factors including growth factors, pro- and anti-inflammatory molecules, and proteases. Coordinated regulation of these factors ensures that the regenerated tissue has the appropriate structure, form and function to fulfil its anatomical and physiological roles.

The repair of damaged tissue is often inefficient because (1) new tissue formation is counteracted by the apoptosis of cells in the vicinity of the tissue damage, (2) an ongoing disease process has a detrimental effect on tissue regeneration, and (3) the local microenvironment is not conducive to tissue repair and regeneration. These limitations often result in a sub-optimal or partial repair of the damaged or senescent tissue.

### DESCRIPTION OF THE FIGURES

FIG. 1 shows oscillation temperature ramp of methylcellulose and PF-127 placebo gels. Storage modulus G′, loss modulus G″ and phase angle δ as a function of temperature for methyl cellulose at 2% (A), 2.5% (B), and 3% (C), and PF-127 at 25% (D), 30% (E) and 35% (F) at 0.2, 0.43, 0.93 and 2.0 Hz. The ramp rate was 2° C. per minute and the torque was 250 μN·m. Gaps in PF-127 gel graphs were due to the low viscosity of the solution at low temperatures.

FIG. 2 shows hotographs of methylcellulose and PF-127 placebo gels. Methyl cellulose placebos (2%, 2.5% and 3%) and PF-127 placebos (20%, 25% and 30%) were stored at 4° C., 22° C. or 37° C. and injected onto an absorbent surface. Water was used as the negative control and Nurofen® ibuprofen gel was used as the positive control. Placebo gels were visually compared to the controls.

FIG. 3 shows oscillation temperature ramp of methyl cellulose and PF-127 therapeutic gels. Storage modulus G′, loss modulus G″ and phase angle δ as a function of temperature for methyl cellulose PL at 2.5% (A), PF-127 PL at 25% (B), and 30% (C) at 0.2, 0.43, 0.93 and 2.0 Hz. The ramp rate was 2° C. per minute and the torque was 250 μN·m. A representative example of 3 gels is shown.

FIG. 4 shows photographs of methyl cellulose and PF-127 PL gels. Methyl cellulose PL (2.5%) and PF-127 PL (25% and 30%) were stored at 4° C., 22° C. or 37° C. and injected onto an absorbent surface. Water was used as the negative control and Nurofen® ibuprofen gel was used as the positive control. PL gels were visually compared to the controls.

FIG. 5 shows fibroblast proliferation with methylcellulose or PF-127 therapeutic gels. Fibroblasts were cultured with 2% methyl cellulose PL (MCPL) or 2% PF-127 PL (PFPL) containing varying concentrations of PL (0, 12.5, 25, 37.5, 50% or 100% [PFPL only]). After 48 hours, an alamarBlue® assay was performed to indicate the relative levels of viable cells. PL (2%) was used as the positive control and cisplatin (15 μM) was used as the negative control. Fluorescent intensity shown is relative to FBS (2%) control. Statistical significance, n=3, *p≦0.05; **p≦0.01; ***p≦0.001.

FIG. 6 shows fibroblast proliferation with methylcellulose or PF-127 therapeutic gels following storage under different conditions. Fibroblasts were cultured with 2% methyl cellulose PL (MCPL) or 2% PF-127 PL (PFPL) which had been stored for 0 days, 7 days, 14 days, 28 days, 2 months, 3 months, or 6 months at room temperature (RT) or 4° C. After 48 hours, an alamarBlue® assay was performed to indicate the relative levels of viable cells. PL (2%) was used as the positive control and cisplatin (15 μM) was used as the negative control. Fluorescent intensity shown is relative to FBS (2%) control. Statistical significance, n=3.

FIG. 7 shows a standard curve generated using five parameter logistic (5-PL) for Example 4.

FIG. 8 shows results for Example 4. FIG. 8A shows the concentration of bFGF, PDGF-BB, VEGF, PDGF-AA, thrombospondin and angiopoeitin in each therapeutic gel analysed. Levels of VEGF-D were below the detection limit and are not shown. FIG. 8B summarises the minimum, maximum, median, mean and SEM for each growth factor, calculated across the sample set. FIG. 8C depicts the median concentration of each growth factor, and FIG. 8D presents this information as a box and whisker plot

FIG. 9 shows a standard curve generated using five parameter logistic (5-PL) for Example 5.

FIG. 10 shows results for Example 5. FIG. 10B shows the concentration of bFGF, PDGF-BB, PDGF-AA, thrombospondin and angiopoeitin in each therapeutic gel analysed. FIG. 10A summarises the minimum, maximum, median, mean, SD and SEM for each growth factor, calculated across the sample set. FIG. 10C depicts the median concentration of each growth factor. VEGF and VEGF-D were undetectable.

FIG. 11 compares the growth factor content of fresh and 2-week stored gel. FIG. 11A depicts the median growth factor concentration of both fresh and 2-week stored gel. FIG. 11B shows the average percentage loss of growth factor from fresh therapeutic gel after 2 weeks of storage.

### DESCRIPTION OF THE SEQUENCE LISTING

SEQ ID NO. 1 shows the amino acid sequence of the 18 kDa isoform of human bFGF.
SEQ ID NO. 2 shows the amino acid sequence of the 22 kDa isoform of human bFGF.
SEQ ID NO. 3 shows the amino acid sequence of the 24 kDa isoform of human bFGF.
SEQ ID NO. 4 shows the amino acid sequence of the 34 kDa isoform of human bFGF.
SEQ ID NO. 5 shows the amino acid sequence of human PDGF subunit A.
SEQ ID NO. 6 shows the amino acid sequence of human PDGF subunit B.
SEQ ID NO. 7 shows the amino acid sequence of human PDGF subunit C.
SEQ ID NO. 8 shows the amino acid sequence of human PDGF subunit D.
SEQ ID NO. 9 shows the amino acid sequence of human IGF-1 isoform 1.
SEQ ID NO. 10 shows the amino acid sequence of human IGF-1 isoform 2.
SEQ ID NO. 11 shows the amino acid sequence of human IGF-1 isoform 3.
SEQ ID NO. 12 shows the amino acid sequence of human IGF-2 isoform 1.
SEQ ID NO. 13 shows the amino acid sequence of human IGF-2 isoform 2.
SEQ ID NO. 14 shows the amino acid sequence of human VEGF121.
SEQ ID NO. 15 shows the amino acid sequence of human VEGF145.
SEQ ID NO. 16 shows the amino acid sequence of human VEGF165.
SEQ ID NO. 17 shows the amino acid sequence of human VEGF165b.
SEQ ID NO. 18 shows the amino acid sequence of human VEGF189.
SEQ ID NO. 19 shows the amino acid sequence of human VEGF206.

### SUMMARY OF THE INVENTION

The inventors have surprisingly found that a composition comprising at least one basic fibroblast growth factor (bFGF) isoform and at least one platelet-derived growth factor (PDGF) isoform promotes effective repair of damaged and senescent tissue. The composition can therefore be used as a therapeutic. The composition also be used to promote hair growth. The composition further be used in a method of producing therapeutic cells.

The invention therefore provides a composition comprising at least one basic fibroblast growth factor (bFGF) isoform and at least one platelet-derived growth factor (PDGF) isoform.

The invention also provides:

a method of repairing a damaged tissue in a patient, comprising administering to the patient a therapeutically effective amount of the composition of the invention, and thereby treating the damaged tissue in the patient;

a method of inhibiting senescence in a patient, comprising administering to the patient a therapeutically effective amount of the composition of the invention, and thereby inhibiting the senescence in the patient;

a method of promoting hair growth in a patient, comprising administering to the patient a therapeutic amount of the composition of the invention;

a composition of the invention for use in a method of repairing damaged tissue in a patient, the method comprising administering to the patient a therapeutically effective amount of the composition;

a composition of the invention for use in a method of inhibiting senescence in a patient, the method comprising administering to the patient a therapeutically effective amount of the composition;

a composition of the invention for use in a method of promoting hair growth ion a patient, the method comprising administering to the patient a therapeutically effective amount of the composition; and a method of producing a population of cells for use in a method of treating damaged tissue or inhibiting senescence, or promoting hair growth, the method comprising culturing mononuclear cells (MCs), progenitor cells of mesodermal lineage (PMLs) and/or immunomodulatory progenitor cells (IMPs) in the presence of the composition of the invention, and allowing at least some of the MCs, PMLs and/or IMPs to proliferate.

### DETAILED DESCRIPTION OF THE INVENTION

It is to be understood that different applications of the disclosed products and methods may be tailored to the specific needs in the art. It is also to be understood that the terminology used herein is for the purpose of describing particular embodiments of the invention only, and is not intended to be limiting.

In addition, as used in this specification and the appended claims, the singular forms “a”, “an”, and “the” include plural referents unless the content clearly dictates otherwise. Thus, for example, reference to “a cell” includes two or more such cells, reference to “a tissue” includes two or more such tissues, reference to “a patient” includes two or more such patients, and the like.

All publications, patents and patent applications cited herein, whether supra or infra, are hereby incorporated by reference in their entirety.

### Composition of the Invention

The invention provides a composition comprising at least one basic fibroblast growth factor (bFGF) isoform and at least one platelet-derived growth factor (PDGF) isoform. The composition may comprise at least 2, at least 3, at least 4 or at least 5 bFGF isoforms. The composition may comprise at least 2, at least 3 or at least 4 PDGF isoforms.

The composition is suitable for repairing damaged tissue. The composition may be contacted with, applied to or injected into damaged tissue. The composition is also suitable for inhibiting senescence. The composition may be contacted with, applied to or injected into senescent tissue. The composition is also suitable for promoting hair growth.

### Basic Fibroblast Growth Factor

Basic fibroblast growth factor (also known as bFGF, FGF2 or FGF-β) is a member of the fibroblast growth factor family. The fibroblast growth factor family is a large family of secreted growth factors whose members have functions in wound healing, angiogenesis, endocrine signaling and multiple developmental processes. They are important for the proliferation and differentiation a large number of cells and tissues. All fibroblast growth factors share a similar internal core, and have a high affinity for heparin. Fibroblast growth factors act through binding and activating fibroblast growth factor receptors, which are tyrosine kinase receptors containing three immunoglobulin-like domains and a heparin-binding sequence. Heparan sulfate proteoglycans on the cell surface also play a role in the transduction of fibroblast growth factor signals.

Like many of the fibroblast growth factors, bFGF has pleiotropic biological roles. It is particularly involved in embryogenesis and morphogenesis in the nervous system, and in bone formation. Basic fibroblast growth factor is also a major angiogenic factor, and has a critical role in wound healing. Basic fibroblast growth factor is present in basement membranes and in the subendothelial extracellular matrix of blood vessels in normal tissue. During wound healing, it is thought that the action of heparan sulfate-degrading enzymes activates bFGF in order to initiate angiogenesis.

In some instances, bFGF acts in a paracrine or autocrine fashion. In other instances, bFGF exhibits intracrine activity. In other words, bFGF can have direct effects on intracellular targets in the absence of secretion.

The at least one bFGF isoform is typically human. Alternatively, the at least one bFGF isoform may be derived from other animals or mammals, for instance from commercially farmed animals, such as horses, cattle, sheep or pigs, from laboratory animals, such as mice or rats, or from pets, such as cats, dogs, rabbits or guinea pigs.

The different modes of action of human bFGF can be attributed to the fact that five different bFGF isoforms can be formed from the single FGF2 gene. The bFGF isoforms all have the potential to perform the same function, but tend to be found in different locations. Each isoform is associated with a different mRNA translation initiation site. An AUG initiation codon leads to an 18 kDa (155 amino acid) cytosolic isoform, which is responsible for autocrine and paracrine effects. Four CUG initiation codons give rise to bFGF isoforms of 22 kDa (196 amino acids), 22.5 (201 amino acids), 24 kDa (210 amino acids), and 34 kDa (288 amino acid). These isoforms localise to the nucleus and are responsible for the intracrine effects of bFGF.

The composition of the invention may comprise any bFGF isoform. The composition of the invention preferably comprises at least one of (a) 18 kDa bFGF (SEQ ID NO: 1) or a variant thereof, (b) 22 kDa bFGF (SEQ ID NO: 2) or a variant thereof, (c) 22.5 kDa bFGF or a variant thereof, (d) 24 kDa bFGF (SEQ ID NO: 3) or a variant thereof and (e) 34 kDa bFGF (SEQ ID NO: 4) or a variant thereof. The composition may comprise any number and any combination of bFGF isoforms. The composition may comprise all of the bFGF isoforms.

The composition of the invention may comprise (a); (b); (c); (d); (e); (a) and (b); (a) and (c); (a) and (d); (a) and (e); (b) and (c); (b) and (d); (b) and (e); (c) and (d); (c) and (e); (d) and (e); (a), (b) and (c); (a), (b) and (d); (a), (b) and (e); (a), (c) and (d); (a), (c) and (e); (a), (d) and (e); (b), (c) and (d); (b), (c) and (e); (b), (d) and (e); (c), (d) and (e); (a), (b), (c) and (d); (a), (b), (c) and (e); (a), (b), (d) and (e); (a), (c), (d) and (e); (b), (c), (d) and (e); or (a), (b), (c), (d) and (e). The combinations for each definition of (a) to (e) are independently selectable from this list.

A variant of a bFGF isoform is a polypeptide that has an amino acid sequence which varies from that of the bFGF isoform and which retains at least partial bFGF activity. The activity of the variant may be decreased compared with the activity of the bFGF isoform from which is derived by at least 5%, at least 10%, at least 20%, at least 30%, at least 40% or at least 50%. A variant of a bFGF isoform is preferably a polypeptide that has an amino acid sequence which varies from that of the bFGF isoform and which retains bFGF activity. bFGF activity can be determined using any method known in the art. For example, the effect of adding a bFGF variant to an in vitro angiogenesis assay could be studied. Angiogenesis assays are known in the art (Auerback et al., Clin Chem. 2003 January; 49(1):32-40).

Over the entire length of the amino acid sequence of the bFGF isoform, such as SEQ ID NO: 1, 2, 3 or 4, a variant will preferably be at least 50% homologous to that sequence based on amino acid identity, such as 50% identical to that sequence. More preferably, the variant may be at least 55%, at least 60%, at least 65%, at least 70%, at least 75%, at least 80%, at least 85%, at least 90% and more preferably at least 95%, 97% or 99% homologous based on amino acid identity to the amino acid sequence of the bFGF isoform over the entire sequence or identical to the amino acid sequence of the bFGF isoform over the entire sequence. There may be at least 80%, for example at least 85%, 90% or 95%, amino acid identity over a stretch of 100 or more, for example 125, 150, 175 or 200 or more, contiguous amino acids (“hard homology”).

Standard methods in the art may be used to determine homology. For example the UWGCG Package provides the BESTFIT program which can be used to calculate homology, for example used on its default settings (Devereux et al (1984) Nucleic Acids Research 12, p 387-395). The PILEUP and BLAST algorithms can be used to calculate homology or line up sequences (such as identifying equivalent residues or corresponding sequences (typically on their default settings)), for example as described in Altschul S. F. (1993) J Mol Evol 36:290-300; Altschul, S. F et al (1990) J Mol Biol 215:403-10. Software for performing BLAST analyses is publicly available through the National Center for Biotechnology Information (http://www.ncbi.nlm.nih.gov/).

Amino acid substitutions may be made to the amino acid sequence of the bFGF isoform, for example up to 1, 2, 3, 4, 5, 10, 20 or 30 substitutions. Conservative substitutions replace amino acids with other amino acids of similar chemical structure, similar chemical properties or similar side-chain volume. The amino acids introduced may have similar polarity, hydrophilicity, hydrophobicity, basicity, acidity, neutrality or charge to the amino acids they replace. Alternatively, the conservative substitution may introduce another amino acid that is aromatic or aliphatic in the place of a pre-existing aromatic or aliphatic amino acid.

Conservative amino acid changes are well-known in the art and may be selected in accordance with the properties of the 20 main amino acids as defined in Table 1 below. Where amino acids have similar polarity, this can also be determined by reference to the hydropathy scale for amino acid side chains in Table 2.

TABLE 1
Chemical properties of amino acids

| | | | |
|---|---|---|---|
| Ala | aliphatic, hydrophobic, neutral | Met | hydrophobic, neutral |
| Cys | polar, hydrophobic, neutral | Asn | polar, hydrophilic, neutral |
| Asp | polar, hydrophilic, charged (−) | Pro | hydrophobic, neutral |
| Glu | polar, hydrophilic, charged (−) | Gln | polar, hydrophilic, neutral |
| Phe | aromatic, hydrophobic, neutral | Arg | polar, hydrophilic, charged (+) |
| Gly | aliphatic, neutral | Ser | polar, hydrophilic, neutral |
| His | aromatic, polar, hydrophilic, charged (+) | Thr | polar, hydrophilic, neutral |
| Ile | aliphatic, hydrophobic, neutral | Val | aliphatic, hydrophobic, neutral |
| Lys | polar, hydrophilic, charged(+) | Trp | aromatic, hydrophobic, neutral |
| Leu | aliphatic, hydrophobic, neutral | Tyr | aromatic, polar, hydrophobic |

TABLE 2
Hydropathy scale

| Side Chain | Hydropathy |
|---|---|
| Ile | 4.5 |
| Val | 4.2 |
| Leu | 3.8 |
| Phe | 2.8 |
| Cys | 2.5 |
| Met | 1.9 |
| Ala | 1.8 |
| Gly | −0.4 |
| Thr | −0.7 |
| Ser | −0.8 |
| Trp | −0.9 |
| Tyr | −1.3 |
| Pro | −1.6 |
| His | −3.2 |
| Glu | −3.5 |
| Gln | −3.5 |
| Asp | −3.5 |
| Asn | −3.5 |
| Lys | −3.9 |
| Arg | −4.5 |

One or more amino acid residues of the amino acid sequence of the bFGF isoform may additionally be deleted from the polypeptides described above. Up to 1, 2, 3, 4, 5, 10, 20 or 30 residues may be deleted, or more.

Variants may include fragments of the bFGF isoform. Such fragments retain at least partial bFGF activity. Fragments may be at least 50, 100, 150 or 200 amino acids in length.

One or more amino acids may be alternatively or additionally added to the polypeptides described above. An extension may be provided at the amino terminal or carboxy terminal of the amino acid sequence of the bFGF isoform or the variant thereof. The extension may be quite short, for example from 1 to 10 amino acids in length. Alternatively, the extension may be longer, for example up to 50 or 100 amino acids.

Basic fibroblast growth factor is secreted as a monomer, but can form dimers upon interaction with cell surface heparan sulfate. In nature, dimeric bFGF has a non-covalent side-to-side configuration that is capable of dimerizing and activating fibroblast growth factor receptors.

The composition of the invention comprises at least one isoform of bFGF as set out above. The at least one bFGF isoform may be monomeric. The at least one bFGF isoform may be dimeric. If the composition comprises at least two bFGF isoforms, all of the isoforms may be monomeric. Alternatively, all of the isoforms may be dimeric. If the composition comprises at least two bFGF isoforms, the composition may comprise both monomeric bFGF and dimeric bFGF. The at least one dimeric bFGF may be homodimeric or heterodimeric. SEQ ID NOs: 1 to 5 and variants thereof are described above as (a) to (e). Homodimeric bFGF may comprise (a) and (a), (b) and (b), (c) and (c), (d) and (d) or (e) and (e). Heterodimeric bFGF may comprise any two different bFGF isoforms or variants thereof. Heterodimeric bFGF may comprise any two different bFGF isoforms or variants thereof described above as (a) to (e). Heterodimeric bFGF preferably comprises (a) and (b); (a) and (c); (a) and (d); (a) and (e); (b) and (c); (b) and (d); (b) and (e); (c) and (d); (c) and (e); or (d) and (e). The composition of the invention comprises at least one of (i) monomeric bFGF, (ii) homodimeric bFGF, and (iii) heterodimeric bFGF, such as (i); (ii); (iii); (i) and (ii); (i) and (iii); (ii) and (iii); or (i), (ii) and (iii). The composition may comprise all of monomeric bFGF, homodimeric bFGF, and heterodimeric bFGF. The composition may comprise more than one form of heterodimeric FGF, such as 2, 3, 4, 5, 6, 7, 8, 9 or 10 forms of heterodimeric FGF.

The at least one bFGF is preferably recombinant bFGF. Methods of producing recombinant proteins are well known in the art.

The total concentration of bFGF polypeptide (i.e. the total amount of the at least one bFGF isoform) in the composition is preferably within in the range of about 50 pg and about 500 pg per milliliter of composition. For example, the total concentration of bFGF polypeptide (i.e. the total amount of the at least one bFGF isoform) may be from about 75 pg to about 475 pg, about 100 pg to about 450 pg, about 125 pg to about 425 pg, about 150 pg to about 400 pg, about 175 pg to about 375 pg, about 200 pg to 350 pg, about 225 pg to about 325 pg, about 250 pg to about 300 pg, or about 275 pg per milliliter of composition.

### Platelet Derived Growth Factor

Like bFGF, platelet derived growth factor (PDGF) regulates cell growth and division. It is produced by platelets, stored in their alpha granules and released upon platelet activation. PDGF is also produced by many other cells including smooth muscle cells, activated macrophages, and endothelial cells.

PDGF stimulates the growth, survival and motility of mesenchymal cells and certain other cell types. This growth factor is particularly important for embryonic development, and for tissue homeostasis in the adult. PDGF also plays a significant role in the regulation of angiogenesis. PDGF isoforms bind to α- and β-tyrosine kinase receptors (PDGFRα and PDGFRβ) to exert their cellular effects. PDGFRα and PDGFRPβ share a similar structure, comprising extracellular domains with five immunoglobulin-like domains and intracellular portions possessing kinase domains.

The at least one PDGF isoform is typically human PDGF. Alternatively, the at least one PDGF isoform may be derived from other animals or mammals, for instance from commercially farmed animals, such as horses, cattle, sheep or pigs, from laboratory animals, such as mice or rats, or from pets, such as cats, dogs, rabbits or guinea pigs.

There are five known isoforms of human PDGF. All of the isoforms are disulphide-linked dimeric proteins. PDGF-AA, PDGF-BB, PDGF-CC and PDGF-DD are homodimers, consisting of two A- (SEQ ID NO: 5), B- (SEQ ID NO: 6), C- (SEQ ID NO: 7) or D- (SEQ ID NO: 8) polypeptide chains respectively. PDGF may also exist as a heterodimer, PDGF-AB. While PDGF-A and PDGF-B are activated intracellularly and subsequently secreted, PDGF-C and PDGF-D are secreted as latent factors that require activation by extracellular proteases. Variation in function exists between the different isoforms.

The composition of the invention comprises at least one PDGF isoform. The composition preferably comprises at least two, at least three, at least four or at least five isoforms of PDGF. The composition preferably comprises at least one homodimeric PDGF. Homodimeric PDGF may comprise (a) two PDGF-A subunits (SEQ ID NO: 5) or a variant thereof, (b) two PDGF B subunits (SEQ ID NO: 6) or a variant thereof, (c) two PDGF-C subunits (SEQ ID NO: 7) or a variant thereof, or (d) two PDGF-D subunits (SEQ ID NO: 8) or a variant thereof. The composition preferably comprises at least one heterodimeric PDGF. Heterodimeric PDGF may comprise (e) one PDGF-A subunit (SEQ ID NO: 5) or a variant thereof and one PDGF-B subunit (SEQ ID NO: 6) or a variant thereof.

The composition may comprise any number of homodimeric and heterodimeric PDGF isoforms, in any combination. The composition may comprise all of the homodimeric and heterodimeric PGDF isoforms. In other words, the composition of the invention may comprise (a); (b); (c); (d); (e); (a) and (b); (a) and (c); (a) and (d); (a) and (e); (b) and (c); (b) and (d); (b) and (e); (c) and (d); (c) and (e); (d) and (e); (a), (b) and (c); (a), (b) and (d); (a), (b) and (e); (a), (c) and (d); (a), (c) and (e); (a), (d) and (e); (b), (c) and (d); (b), (c) and (e); (b), (d) and (e); (c), (d) and (e); (a), (b), (c) and (d); (a), (b), (c) and (e); (a), (b), (d) and (e); (a), (c), (d) and (e); (b), (c), (d) and (e); or (a), (b), (c), (d) and (e). The combinations for each definition of (a) to (e) are independently selectable from this list.

A variant of a PDGF isoform is a polypeptide that has an amino acid sequence which varies from that of the PDGF isoform and which retains at least partial PDGF activity. The activity of the variant may be decreased compared with the activity of the PDGF isoform from which is derived by at least 5%, at least 10%, at least 20%, at least 30%, at least 40% or at least 50%. A variant of a PDGF isoform is preferably a polypeptide that has an amino acid sequence which varies from that of the PDGF isoform and which retains PDGF activity. PDGF activity can be determined using any method known in the art. For example, the effect of adding a PDGF variant to an in vitro angiogenesis assay could be studied. Angiogenesis assays are known in the art (Auerback et al., Clin Chem. 2003 January; 49(1):32-40).

Over the entire length of the amino acid sequence of the PDGF isoform, such as SEQ ID NO: 5, 6, 7 or 8 a variant will preferably be at least 50% homologous to that sequence based on amino acid identity, such as 50% identical to that sequence. More preferably, the variant may be at least 55%, at least 60%, at least 65%, at least 70%, at least 75%, at least 80%, at least 85%, at least 90% and more preferably at least 95%, 97% or 99% homologous based on amino acid identity to the amino acid sequence of the PDGF isoform over the entire sequence or identical to the amino acid sequence of the PDGF isoform over the entire sequence. There may be at least 80%, for example at least 85%, 90% or 95%, amino acid identity over a stretch of 100 or more, for example 125, 150, 175 or 200 or more, contiguous amino acids (“hard homology”).

The PDGF is preferably recombinant PDGF. Methods of producing recombinant proteins are well known in the art.

The total concentration of PDGF polypeptide (i.e. the total amount of the at least one PDGF isoform) in the composition of the invention is preferably within in the range of about 5000 pg and about 25000 pg per milliliter of composition. For example, the total concentration of PDGF polypeptide (i.e. the total amount of the at least one PDGF isoform) may be from about 6000 pg to about 24000 pg, about 7000 pg to about 23000 pg, about 8000 pg to about 22000 pg, about 9000 pg to about 21000 pg, about 10000 pg to about 20000 pg, about 11000 pg to about 19000 pg, about 12000 pg to about 18000 pg, about 13000 pg to about 17000 pg, about 14000 pg to about 16000 pg, about 15000 pg per milliliter of composition.

The ratio of total bFGF polypeptide to total PDGF polypeptide in the composition is within the range of about 1:10 and about 1:500, such as from about 1:50 to about 1:450, from about 1:100 to about 1:400, from about 1:150 to about 1:350, or from about 1:200 to about 1:300.

### Other Growth Factors and Bioactive Components

The bFGF and PDGF in the composition of the invention promote survival, repair and regeneration of the neighbouring cells in the damaged tissue. They also promote angiogenesis. Accordingly, the composition of the invention preferably increases or improves tissue regeneration and/or decreases or reduces apoptosis. The composition preferably increases or improves tissue regeneration compared with the absence of the composition.

In addition to bFGF and PDGF, the composition of the invention may comprise detectable levels of one or more of other growth factors or bioactive components. Levels of the one or more other growth factors or bioactive components may be measured using known techniques, as described below.

As for bFGF and PDGF above, the growth factors or other bioactive components discussed below are typically human. Alternatively, they may be derived from other animals or mammals, for instance from commercially farmed animals, such as horses, cattle, sheep or pigs, from laboratory animals, such as mice or rats, or from pets, such as cats, dogs, rabbits or guinea pigs.

The composition preferably comprises detectable levels of at least one, such as at least 2, at least 3, at least 4, at least 5, at least 6, at least 7, at least 8, at least 9, at least 10, at least 11, at least 12, at least 13, at least 14, at least 15, at least 16, at least 17, at least 18 or at least 19, of angiopoietin, CXCL4, CXCL7, CXCL12, epidermal growth factor (EGF), interleukin 4 (IL-4), IL-8, IL-13, IL-17, interferon α (IFNα), lipoxin A4 (LXA4), protease-activated receptor 4 (PAR4), chemokine (C-C motif ligand) 5 (CCL5/RANTES), PDGF-AA, PDGF-AB, transforming growth factor beta 1 (TGF-β1), TGF-β2, thrombospondin, tumour necrosis factor alpha (TNFα) and vascular endothelial growth factor D (VEGF-D). The composition may comprise detectable levels of any number and combination of these growth factors or bioactive components. The composition may comprise detectable levels of all of these components.

The composition preferably comprises detectable levels of at least one of (i) angiopoietin, (ii) CXCL4, (iii) CXCL7, (iv) CXCL12, (v) lipoxin A4 (LXA4), (vi) protease-activated receptor 4 (PAR4) and (vii) thrombospondin. The composition may comprise detectable levels of any number and combination of these growth factors or bioactive components. The composition may comprise detectable levels of all of these components. Specifically, the composition may comprise detectable levels of (i); (ii); (iii); (iv); (v); (vi); (vii); (i) and (ii); (i) and (iii); (i) and (iv); (i) and (v); (i) and (vi); (i) and (vii); (ii) and (iii); (ii) and (iv); (ii) and (v); (ii) and (vi); (ii) and (vii); (iii) and (iv); (iii) and (v); (iii) and (vi); (iii) and (vii); (iv) and (v); (iv) and (vi); (iv) and (vii); (v) and (vi); (v) and (vii); (vi) and (vii); (i), (ii) and (iii); (i), (ii) and (iv); (i), (ii) and (v); (i), (ii) and (vi); (i), (ii) and (vii); (i), (iii) and (iv); (i), (iii) and (v); (i), (iii) and (vi); (i), (iii) and (vii); (i), (iv) and (v); (i), (iv) and (vi); (i), (iv) and (vii); (i), (v) and (vi); (i), (v) and (vii); (i), (vi) and (vii); (ii), (iii) and (iv); (ii), (iii) and (v); (ii), (iii) and (vi); (ii), (iii) and (vii); (ii), (iv) and (v); (ii), (iv) and (vi); (ii), (iv) and (vii); (ii), (v) and (vi); (ii), (v) and (vii); (ii), (vi) and (vii); (iii), (iv) and (v); (iii), (iv) and (vi); (iii), (iv) and (vii); (iii), (v) and (vi); (iii), (v) and (vii); (iii), (vi) and (vii); (iv), (v) and (vi); (iv), (v) and (vii); (iv), (vi) and (vii); (v), (vi) and (vii); (i), (ii), (iii) and (iv); (i), (ii), (iii) and (v); (i), (ii), (iii) and (vi); (i), (ii), (iii) and (vii); (i), (ii), (iv) and (v); (i), (ii), (iv) and (vi); (i), (ii), (iv) and (vii); (i), (ii), (v) and (vi); (i), (ii), (v) and (vii); (i), (ii), (vi) and (vii); (i), (iii), (iv) and (v); (i), (iii), (iv) and (vi); (i), (iii), (iv) and (vii); (i), (iii), (v) and (vi); (i), (iii), (v) and (vii); (i), (iii), (vi) and (vii); (i), (iv), (v) and (vi); (i), (iv), (v) and (vii); (i), (iv), (vi) and (vii); (i), (v), (vi) and (vii); (ii), (iii), (iv) and (v); (ii), (iii), (iv) and (vi); (ii), (iii), (iv) and (vii); (ii), (iii), (v) and (vi); (ii), (iii), (v) and (vii); (ii), (iii), (vi) and (vii); (ii), (iv), (v) and (vi); (ii), (iv), (v) and (vii); (ii), (iv), (vi) and (vii); (ii), (v), (vi) and (vii); (iii), (iv), (v) and (vi); (iii), (iv), (v) and (vii); (iii), (iv), (vi) and (vii); (iii), (v), (vi) and (vii); (iv), (v), (vi) and (vii); (i), (ii), (iii), (iv) and (v); (i), (ii), (iii), (iv) and (vi); (i), (ii), (iii), (iv) and (vii); (i), (ii), (iii), (v) and (vi); (i), (ii), (iii), (v) and (vii); (i), (ii), (iii), (vi) and (vii); (i), (ii), (iv), (v) and (vi); (i), (ii), (iv), (v) and (vii); (i), (ii), (iv), (vi) and (vii); (i), (ii), (v), (vi) and (vii); (i), (iii), (iv), (v) and (vi); (i), (iii), (iv), (v) and (vii); (i), (iii), (iv), (vi) and vii); (i), (iii), (v), (vi) and (vii); (i), (iv), (v), (vi) and (vii); (ii), (iii), (iv), (v) and (vi); (ii), iii), (iv), (v) and (vii); (ii), (iii), (iv), (vi) and (vii); (ii), (iii), (v), (vi) and (vii); (ii), (iv), (v), (vi) and (vii); (iii), (iv), (v), (vi) and vii); (i), (ii), (iii), (iv), (v) and (vi); (i), (ii), (iii), (iv), (v) and (vii); (i), (ii), (iii), (iv), (vi) and (vii); (i), (ii), (iii), (v), (vi) and (vii); (i), (ii), (iv), (v), (vi) and (vii); (i), (iii), (iv), (v), (vi) and (vii); (ii), (iii), (iv), (v), (vi) and (vii); or (i), (ii), (iii), (iv), (v), (vi) and (vii). The combinations for each definition of (i) to (vii) are independently selectable from this list.

Preferably, the composition comprises a detectable level of angiopoietin.

The composition of the invention preferably comprises detectable levels of at least one bFGF isoform, PDGF-AB and PDGF-BB. More preferably, the composition may comprise any or all of (a) EGF, (b) TGF-β1, (c) TGF-β2, (d) insulin-like growth factor (IGF) and (e) vascular endothelial growth factor (VEGF). In other words, the composition of the invention may comprise (a); (b); (c); (d); (e); (a) and (b); (a) and (c); (a) and (d); (a) and (e); (b) and (c); (b) and (d); (b) and (e); (c) and (d); (c) and (e); (d) and (e); (a), (b) and (c); (a), (b) and (d); (a), (b) and (e); (a), (c) and (d); (a), (c) and (e); (a), (d) and (e); (b), (c) and (d); (b), (c) and (e); (b), (d) and (e); (c), (d) and (e); (a), (b), (c) and (d); (a), (b), (c) and (e); (a), (b), (d) and (e); (a), (c), (d) and (e); (b), (c), (d) and (e); or (a), (b), (c), (d) and (e). The combinations for each definition of (a) to (e) are independently selectable from this list.

The IGF is preferably IGF-1. The IGF-1 is preferably human IGF-1. The composition may comprise detectable levels of any or all of IGF-1 isoform 1 (SEQ ID NO: 9) or a variant thereof, IGF-1 isoform 2 (SEQ ID NO: 10) or a variant thereof, IGF-1 isoform 3 (SEQ ID NO: 11) or a variant thereof, or IGF-1 isoform 4 or a variant thereof. The IGF is preferably IGF-2. The IGF is preferably human IGF-2. The composition may comprise any or all of IGF-2 isoform 1 (SEQ ID NO: 12) or a variant thereof, IGF-2 isoform 2 (SEQ ID NO: 13) or a variant thereof, IGF-1 isoform 3. The composition may comprise detectable levels of at least one IGF-1 isoform and at least one IGF-2 isoform. A variant of an IGF isoform is a polypeptide that has an amino acid sequence which varies from that of the IGF isoform and which retains at least partial IGF activity. Methods for evaluating IGF activity are known in the art. Characteristics of variants, such as % homology or identity, are set out above in relation to bFGF and PDGF and are equally applicable to variants of IGF.

The VEGF is preferably human VEGF. The VEGF may be VEGF121 (SEQ ID NO: 14) or a variant thereof, VEGF145 (SEQ ID NO: 15) or a variant thereof, VEGF165 (SEQ ID NO: 16) or a variant thereof, VEGF165b (SEQ ID NO: 17) or a variant thereof, VEGF189 (SEQ ID NO: 18) or a variant thereof or VEGF206 (SEQ ID NO: 19) or a variant thereof. The composition may comprise any or all of VEGF121, VEGF145, VEGF165, VEGF165b, VEGF189 or VEGF206. A variant of an VEGF isoform is a polypeptide that has an amino acid sequence which varies from that of the VEGF isoform and which retains at least partial VEGF activity. Methods for evaluating VEGF activity are known in the art. Characteristics of variants, such as % homology or identity, are set out above in relation to bFGF and PDGF and are equally applicable to variants of VEGF.

The composition may comprise any or all of (A) transforming growth factor alpha (TGF-α), (B) acidic fibroblast growth factor (aFGF), (C) TGF-β3 and (D) FGF-7. For instance, the composition may comprise (A); (B); (C); (D); (A) and (B); (A) and (C); (A) and (D); (B) and (C); (B) and (D); (C) and (D); (A), (B) and (C); (A), (B) an (D); (A), (C) and (D); (B), (C) and (D); or (A), (B), (C) and (D). The combinations for each definition of (A) to (D) are independently selectable from this list.

The composition may comprise all of angiopoietin, CXCL4, CXCL7, CXCL12, EGF, IL-4, IL-8, L-13, IL-17, IFNα, LXA4, PAR4, CCL5/RANTES, PDGF-AA, PDGF-AB, TGF-β1, TGF-β2, thrombospondin, TNFα, VEGF-D, bFGF isoform, PDGF-BB, IGF, VEGF, TGF-α, aFGF), TGF-β3 and FGF-7.

The composition of the invention may lack detectable levels of at least one other growth factor or bioactive component, such as at least any of 2 to 91 other growth factors or bioactive components inclusive. For instance, the composition of the invention preferably does not comprise detectable levels of placental growth factor (PlGF). The composition preferably does not comprise detectable levels of at least one of activin A, activin C, activin β, artemin, brain-derived neurotrophic factor (BDNF), bone morphogenetic protein 15 (BMP-15), BMP-2, BMP3, BMP-3b, BMP-4, BMP-5, BMP-6, BMP-7, BMP-8, beta nerve growth factor (b-NGF), betacellulin (BTC), ciliary neurotrophic factor (CNTF), connective tissue growth factor (CTGF), Dickkopf-related protein 1 (Dkk-1), endocan, eotaxin, epiregulin, fibroblast growth factor 10 (FGF-10), FGF-11, FGF-12, FGF-13, FGF-16, FGF-17, FGF-18, FGF-19, FGF-20, FGF-21, FGF-23, FGF-4, FGF-6, FGF-8, FGF-9, granulocyte colony-stimulating factor (GCSF), growth differentiation factor 1 (GDF-1), GDF-11, GDF-15, GDF-3, GDF-5, GDF-8, GDF-9, glial cell-derived neurotrophic factor (GDNF), growth hormone 1 (GH-1), granulocyte macrophage colony-stimulating factor (GM-CSF), chemokine (C-X-C motif) ligand 1 (CXCL1), CXCL2, CXCL3, Heparin-binding EGF-like growth factor (HB-EGF), hepatocyte growth factor (HGF), interferon γ (IFNγ), insulin-like growth factor 1 (IGF-1), IGF-11, interleukin 10 (IL-10), IL-12, IL-15, IL-1a, IL-1b, IL-1RA, IL-1β, IL-2, IL-2R, L-5, IL-6, IL-7, inhibin A, interferon gamma-induced protein 10 (IP-10), lefty A, leukaemia inhibitory factor (LIF), monocyte chemotactic protein 1 (MCP-1), macrophage colony-stimulating factor (M-CSF), milk fat globule-EGF factor 8 protein (MFG-E8), MIG, macrophage inflammatory proteins (MIP-1a), MIP-1b, neurturin, nephroblastoma overexpressed gene (NOV), neurotrophin 3 (NT-3), NT-4, platelet-derived growth factor C (PDGF-C), persephin, progranulin, soluble CD40 ligand (sCD40L), Skp, Cullin, F-box containing complex (SCF) and soluble vascular cell adhesion molecule 1 (sVCAM-1). Any number and combination of these growth factors or bioactive components may be absent at detectable levels from the composition. The composition may lack detectable levels of all of these components.

### Pharmaceutically Acceptable Polymer

The composition of the invention preferably comprises at least one pharmaceutically acceptable polymer. The composition may comprise any number of pharmaceutically acceptable polymers, such as 1, 2, 3, 4, 5, 10 or more.

A polymer is pharmaceutically acceptable if it is suitable for use in therapy. The polymer is preferably suitable for localised administration to a damaged tissue, such as tendon, ligament, cardiac, bone, cartilage, liver, kidney, lung tissue or vaginal tissue. The polymer is preferably suitable for topical administration for the promotion of hair growth or for the treatment of vaginal atrophy.

Pharmaceutically acceptable polymers are well known in the art. Any such polymers may be used in accordance with the invention. Suitable polymers include, but are not limited to, alginate polymers, double ester polymers of ethylidene, the copolymer poly(D,L-lactide-co-glycolide) (PLGA), poly(vinyl alcohol) (PVA), the copolymer polyperfluorooctyloxycaronyl-poly(lactic acid) (PLA-PFO) and other block copolymers. Block copolymers are polymeric materials in which two or more monomer sub-units that are polymerized together to create a single polymer chain. Block copolymers typically have properties that are contributed by each monomer sub-unit. However, a block copolymer may have unique properties that polymers formed from the individual sub-units do not possess. Block copolymers can be engineered such that one of the monomer sub-units is hydrophobic (i.e. lipophilic), whilst the other sub-unit(s) are hydrophilic whilst in aqueous media. In this case, the block copolymer may possess amphiphilic properties and may form a structure that mimics a biological membrane. The block copolymer may be a diblock (consisting of two monomer sub-units), but may also be constructed from more than two monomer sub-units to form more complex arrangements that behave as amphipiles. The copolymer may be a triblock, tetrablock or pentablock copolymer. Block copolymers may also be constructed from sub-units that are not classed as lipid sub-materials; for example a hydrophobic polymer may be made from siloxane or other non-hydrocarbon based monomers. The hydrophilic sub-section of block copolymer can also possess low protein binding properties, which allows the creation of a membrane that is highly resistant when exposed to raw biological samples. This head group unit may also be derived from non-classical lipid head-groups.

The polymer concentration is preferably from about 15% (w/w) to about 30% (w/w), such as from about 17% (w/w) to about 25% (w/w) or from about 20% (w/w) to about 23% (w/w).

The polymer is preferably a cellulose polymer. Suitable cellulose polymers are know in the art. The cellulose polymer is preferably carboxymethylcellulose, hydroxypropylmethylcellulose or methylcellulose. The cellulose polymer concentration is preferably from about 1.5% (w/w) to about 4.0% (w/w), such as from about 2.0% (w/w) to about 3.0% (w/w). The cellulose polymer preferably has a molecular weight of from about 450,000 to about 4,000,000, such as from about 500,000 to about 3,500,000, from about 500,000 to about 3,000,000 or from about 750,000 to about 2,500,000 or from about 1,000,000 to about 2,000,000.

The polymer is preferably a pluronic acid, optionally Pluronic F-127.

The polymer is preferably a gel. Suitable gels are known in the art. The biocompatible gel may be natural or synthetic. Preferred gels include, but are not limited to, a cellulose gel, a collagen gel, a gelatin gel, a fibrin gel, a chitosan gel, a starch gel, an alginate gel, a hyaluronan gel, an agarose gel, a poloaxmer gel or a combination thereof.

The polymer is typically biocompatible. A polymer is biocompatible if it does not cause any adverse reactions or side effects when contacted with a damaged tissue.

### Viscosity

The composition of the invention is preferably a gel as set out above. The composition of the invention is preferably a hydrogel.

The gel has a viscosity in the range of from about 1000 to about 500,000 micropascal-second (mPa·s) (also known as centipoises; cps) at room temperature. Viscosity is a measure of the resistance of the gel to being deformed by either shear stress or tensile stress. Viscosity can be measured using any method known in the art. Suitable methods include, but are not limited to, using a viscometer or a rheometer.

Room temperature is typically from about 18° C. to about 25° C., such as from about 19° C. to about 24° C. or from about 20° C. to about 23° C. or from about 21° C. to about 22° C. Room temperature is preferably any of 18° C., 19° C., 20° C., 21° C., 22° C., 23° C., 24° C. and 25° C. Viscosity is most preferably measured at 25° C.

The gel preferably has a viscosity in the range of from about 1000 to about 500,000 mPa·s at room temperature, such as from about 1500 to about 450,000 mPa·s at room temperature, from about 2000 to about 400,000 mPa·s at room temperature, from about 2500 to about 350,000 mPa·s at room temperature, from about 5000 to about 300,000 mPa·s at room temperature, from about 10,000 to about 250,000 mPa·s at room temperature, from about 50,000 to about 200,000 mPa·s at room temperature or from about 50,000 to about 150,000 mPa·s at room temperature.

The gel most preferably has a viscosity in the range of from about 50,000 to about 150,000 mPa·s (cps) at 25° C.

### Preservatives

The composition of the invention may further comprise one or more preservatives. Suitable preservatives are known in the art. Suitable preservatives include, but are not limited to, methylparaben, propylparaben and m-cresol.

### Osmolality

The composition of the invention preferably has an osmolality in the range of 100 to 500 mOsmol/kg, such as 150 to 450 mOsmol/kg, 200 to 400 mOsmol/kg or 250 to 350 mOsmol/kg. The composition preferably has an osmolality in the range of 280 to 320 mOsmol/kg.

### Xeno-Free

The composition of the invention is preferably xeno-free. A xeno-free composition contains no animal-derived components but may contain human-derived components. The composition may also be devoid of both animal-derived components and human-derived components.

### Therapeutic Cells

The composition preferably comprises one or more therapeutic cells. Therapeutic cells are cells which are capable of having a therapeutic effect. Therapeutic cells are typically living cells. Therapeutic cells are typically cells which are capable of repairing damaged or senescent tissue. The one or more therapeutic cells are preferably autologous. In other words, the one or more cells are preferably derived from the patient into which the cells will be administered to repair damaged tissue, to inhibit senescence or to promote hair growth. Alternatively, the one or more cells are preferably allogeneic. In other words, the cells are preferably derived from a patient that is immunologically compatible with the patient into which the cells will be administered to repair damaged tissue, to inhibit senescence or to promote hair growth. The one or more cells may be semi-allogeneic. Semi-allogeneic populations are typically produced from two or more patients that are immunologically compatible with the patient into which the cells will be administered. In other words, all of the one or more cells are preferably genetically identical with the patient into which they will be administered or sufficiently genetically identical that the cells are immunologically compatible with the patient into which they will be administered.

Any number of cells may be present in the composition. The composition may comprise only one cell.

The composition typically comprises more than one cell, such at least 2, at least 5, at least 10, at least 20, at least 30, at least 40, at least 50, at least 100, at least 200, at least 500, at least 1000, at least 2000, at least 5000, at least 10000, at least 50000, at least 100000, at least 5×10⁵, at least 1×10⁶, at least 2×10⁶, at least 5×10⁶, at least 1×10⁷, at least 2×10⁷, at least 5×10⁷, at least 1×10⁸ or at least 2×10⁸ cells. In some instances, the composition may comprise at least 1.0×10⁷, at least 1.0×10⁸, at least 1.0×10⁹, at least 1.0×10¹⁰, at least 1.0×10¹¹ or at least 1.0×10¹² cells or even more cells.

If more than one cell is present in the composition, the population of cells may be homogenous. In other words, all of the cells in the population may be the same type of cell, e.g. mesenchymal stem cells (MSCs). Alternatively, the population of cells may be heterogeneous. In other words, the population of cells may contain different types of cells, such MSCs and progenitor cells of mesodermal lineage (PMLs).

The one or more therapeutic cells are preferably one or more PMLs. PMLs are disclosed in PCT/GB2012/051600 (published as WO 2013/005053). Any of the cells disclosed therein may be used. The PML expresses detectable levels of CD29, CD44, CD73, CD90, CD105 and CD271, but does not express detectable levels of CD14, CD34 and CD45.

The PMLs may advantageously be used to repair damaged tissues in patients, to inhibit senescence, or to promote hair growth. The PMLs are capable of efficiently exerting anti-inflammatory effects in the tissue. The anti-inflammatory effects of the PMLs promote survival, repair and regeneration of the neighbouring cells in the damaged or senescent tissue. The cells are also able to exert paracrine effects such as the secretion of angiogenic, chemotactic and anti-apoptotic factors.

As discussed in more detail below, the PMLs are produced from mononuclear cells (MCs), such as peripheral MCs, taken from an individual, such as a human individual. Since the PMLs are produced from MCs, they may be produced easily (such as from peripheral blood) and may be autologous for the patient to be treated and thereby avoid the risk of immunological rejection by the patient.

It is possible, in principle, to produce an unlimited number of PMLs from a single individual or patient, since various samples of MCs (i.e. various samples of blood) may be obtained. It is certainly possible to produce very large numbers of PMLs from a single individual or patient. The PMLs of the invention can therefore be made in large numbers.

The PMLs are produced in clinically relevant conditions, for instance in the absence of trace amounts of endotoxins and other environmental contaminants, as well as animal products such as fetal calf serum. This makes the PMLs particularly suitable for administration to patients.

Since the PMLs are produced from MCs, they are substantially homologous and may be autologous. They also avoid donor-to-donor variation, which frequently occurs with MSCs. Numerous populations of PMLs can be produced from a single sample taken from the patient before any other therapy, such as chemotherapy or radiotherapy, has begun. Therefore, the PMLs can avoid any of the detrimental effects of those treatments.

The PMLs can be made quickly. PMLs can be produced from MCs in less than 30 days, such as in about 22 days.

The production of PMLs from MCs avoids the moral and ethical implications involved with using mesenchymal stem cells (MSCs) derived from human embryonic stem cells (hESCs).

The PMLs are typically produced from human MCs. The PMLs are therefore typically human. Alternatively, the PML cells may be produced from MCs from other animals or mammals, for instance from commercially farmed animals, such as horses, cattle, sheep or pigs, from laboratory animals, such as mice or rats, or from pets, such as cats, dogs, rabbits or guinea pigs.

The PMLs can be identified as progenitor cells of mesodermal lineage using standard methods known in the art, including expression of lineage restricted markers, structural and functional characteristics. The PMLs will express detectable levels of cell surface markers known to be characteristic of progenitor cells of mesodermal lineage. In particular, in addition to the markers discussed in more detail below, the PMLs may express α-smooth muscle actin, collagen type I α-chain, GATA6, Mohawk, and vimentin (Sági B et al Stem Cells Dev. 2012 Mar. 20; 21(5):814-28).

The PMLs are capable of successfully completing differentiation assays in vitro to confirm that they are of mesodermal lineage. Such assays include, but are not limited to, adipogenic differentiation assays, osteogenic differentiation assays and neurogenic differentiation assays (Zaim M et al Ann Hematol. 2012 August; 91(8): 1175-86).

The PMLs are not stem cells. In particular, they are not MSCs. They are terminally differentiated. Although they can be forced under the right conditions in vitro to differentiating, for instance into cartilage or bone cells, they do not differentiate in vivo. The PMLs have their effects by exerting paracrine signalling in the damaged tissue. In particular, the PMLs are preferably capable of inducing anti-flammatory effects in the damaged tissue. This is discussed in more detail below.

The PMLs are typically characterised by a spindle-shaped morphology. The PMLs are typically fibroblast-like, i.e. they have a small cell body with a few cell processes that are long and thin. The cells are typically from about 10 to about 20 m in diameter.

The PMLs are distinguished from other cells via their marker expression pattern. The PMLs express detectable levels of CD29, CD44, CD73, CD90, CD105 and CD271. The PMLs may overexpress one or more of, such as all of, CD29, CD44, CD73, CD90, CD105 and CD271. The PMLs overexpress one or more of CD29, CD44, CD73, CD90, CD105 and CD271 if they express more than other PMLs and/or MSCs. The PMLs do not express detectable levels of CD14, CD34 and CD45. The PMLs preferably express CD62E (E-selectin) and/or CD62P (P-selectin).

Standard methods known in the art may be used to determine the detectable expression, low expression or lack thereof of the various markers discussed above (and below). Suitable methods include, but are not limited to, immunocytochemistry, immunoassays, flow cytometry, such as fluorescence activated cells sorting (FACS), and polymerase chain reaction (PCR), such as reverse transcription PCR (RT-PCR). Suitable immunoassays include, but are not limited to, Western blotting, enzyme-linked immunoassays (ELISA), enzyme-linked immunosorbent spot assays (ELISPOT assays), enzyme multiplied immunoassay techniques, radioallergosorbent (RAST) tests, radioimmunoassays, radiobinding assays and immunofluorescence. Western blotting, ELISAs and RT-PCR are all quantitative and so can be used to measure the level of expression of the various markers if present. The use of FACS is preferred. Antibodies and fluorescently-labelled antibodies for all of the various markers discussed herein are commercially-available.

The one or more therapeutic cells are preferably one or more immuno-modulatory progenitor (IMP) cells as disclosed in the UK Application GB 1410504.3. IMP cells express detectable levels of MIC A/B, CD304 (Neuropilin 1), CD178 (FAS ligand), CD289 (Toll-like receptor 9), CD363 (Sphingosine-1-phosphate receptor 1), CD99, CD181 (C-X-C chemokine receptor type 1; CXCR1), epidermal growth factor receptor (EGF-R), CXCR2 and CD126.

The IMP cells may advantageously be used to repair damaged tissues, to inhibit senescence, or to promote hair growth in patients. The IMP cells are capable of exerting anti-inflammatory effects in the tissue. The anti-inflammatory effects of the IMP cells promote survival, repair and regeneration of the neighbouring cells in the damaged or senescent tissue. The cells are also able to exert paracrine effects such as the secretion of angiogenic, chemotactic and anti-apoptotic factors.

The IMP cells are typically produced from human MCs. The IMP cells of the invention are therefore typically human. Alternatively, the IMP cells may be produced from MCs from other animals or mammals, for instance from commercially farmed animals, such as horses, cattle, sheep or pigs, from laboratory animals, such as mice or rats, or from pets, such as cats, dogs, rabbits or guinea pigs.

The IMP cells are also produced from MCs, such as peripheral MCs, taken from an individual or patient, such as a human individual or patient. Hence, they have the same advantages as PMLs discussed above. The IMP cells are capable of successfully completing differentiation assays in vitro to confirm that they are of mesodermal lineage. Such assays include, but are not limited to, adipogenic differentiation assays, osteogenic differentiation assays and neurogenic differentiation assays (Zaim M et al Ann Hematol. 2012 August; 91(8): 1175-86).

The IMP cells are not stem cells. In particular, they are not MSCs. They are terminally differentiated. Although they can be forced under the right conditions in vitro to differentiating, for instance into cartilage or bone cells, they typically do not differentiate in vivo. The IMP cells of the invention have their effects by exerting paracrine signalling in the damaged tissue. In particular, the IMP cells are preferably capable of inducing anti-inflammatory effects in the damaged tissue. This is discussed in more detail below.

The IMP cells are typically characterised by a spindle-shaped morphology. The IMP cells are typically fibroblast-like, i.e. they have a small cell body with a few cell processes that are long and thin. The cells are typically from about 10 to about 20 μm in diameter.

The IMP cells of the invention are distinguished from known cells, including MSCs, via their marker expression pattern. The IMP cells express detectable levels of MIC A/B, CD304 (Neuropilin 1), CD178 (FAS ligand), CD289 (Toll-like receptor 9), CD363 (Sphingosine-1-phosphate receptor 1), CD99, CD181 (C-X-C chemokine receptor type 1; CXCR1), epidermal growth factor receptor (EGF-R), CXCR2 and CD126. The IMP cells preferably express an increased amount of these markers compared with MSCs. This can be determined by comparing the expression level/amount of the markers in an IMP of the invention with the expression level/amount in an MSC using the same technique under the same conditions. Suitable MSCs are commercially available. The MSC used for comparison is preferably a human MSC. Human MSCs are commercially available from Mesoblast® Ltd, Osiris Therapeutics® Inc. or Lonza®. The human MSC is preferably obtained from Lonza®. Such cells were used for the comparison in the Example. The MSC may be derived from any of the animals or mammals discussed above.

The IMP cells preferably express an increased amount of one or more of MIC A/B, CD304 (Neuropilin 1), CD178 (FAS ligand), CD289 (Toll-like receptor 9), CD363 (Sphingosine-1-phosphate receptor 1), CD99, CD181 (C-X-C chemokine receptor type 1; CXCR1), epidermal growth factor receptor (EGF-R), CXCR2 and CD126 compared with a MSC. The IMP cells preferably express an increased amount of all of the ten markers compared with a MSC.

Any of the methods disclosed above may be used to determine the detectable expression or increased expression of these markers. The expression or increased expression of any of the markers disclosed herein is preferably done using high-throughput FACS (HT-FACS).

The IMP cells preferably demonstrate an antibody mean fluorescence intensity (MFI) of at least 330, such as at least 350 or at least 400, for MIC A/B, an MFI of at least 210, such as at least 250 or at least 300, for CD304 (Neuropilin 1), an MFI of at least 221, such as at least 250 or at least 300, for CD178 (FAS ligand), an MFI of at least 186, such as at least 200 or at least 250, for CD289 (Toll-like receptor 9), an MFI of at least 181, such as at least 200 or at least 250, for CD363 (Sphingosine-1-phosphate receptor 1), an MFI of at least 184, such as at least 200 or at least 250, for CD99, an MFI of at least 300, such as at least 350 or at least 400, for CD181 (C-X-C chemokine receptor type 1; CXCR1), an MFI of at least 173, such as at least 200 or at least 250, for epidermal growth factor receptor (EGF-R), an MFI of at least 236, such as at least 250 or at least 300, for CXCR2 and an MFI of at least 160, such as at least 200 or at least 250, for CD126. Mean fluorescent intensity (MFI) is a measure of intensity, time average energy flux measured in watts per square metre. It is an SI unit. The MFI for each marker is typically measured using HT-FACS. The MFI for each marker is preferably measured using HT-FACS as described in the Example of the UK Application being filed concurrently with this application GB 1410504.3.

In addition to the ten markers specified above, the IMP cells typically express detectable levels of one or more of the other markers shown in Table 1 of GB 1410504.3. The IMP cells may express detectable levels of any number and combination of those markers.

The IMP cells preferably express detectable levels of one or more of CD267, CD47, CD51/CD61, CD49f, CD49d, CD146, CD340, Notch2, CD49b, CD63, CD58, CD44, CD49c, CD105, CD166, HLA-ABC, CD13, CD29, CD49e, CD73, CD81, CD90, CD98, CD147, CD151 and CD276. The IMP cells more preferably express detectable levels of one or more of CD10, CD111, CD267, CD47, CD273, CD51/CD61, CD49f, CD49d, CD146, CD55, CD340, CD91, Notch2, CD175s, CD82, CD49b, CD95, CD63, CD245, CD58, CD108, B2-microglobulin, CD155, CD298, CD44, CD49c, CD105, CD166, CD230, HLA-ABC, CD13, CD29, CD49e, CD59, CD73, CD81, CD90, CD98, CD147, CD151 and CD276. The IMP cells may express detectable levels of any number and combination of these markers. The IMP cells preferably express detectable levels of all of these markers.

The IMP cells preferably express detectable levels of one or more of CD156b, CD61, CD202b, CD130, CD148, CD288, CD337, SSEA-4, CD349 and CD140b. The IMP cells more preferably express detectable levels of one or more of CD156b, CD61, CD202b, CD130, CD148, CD288, CD337, SSEA-4, CD349, CD140b, CD10, CD111, CD267, CD47, CD273, CD51/CD61, CD49f, CD49d, CD146, CD55, CD340, CD91, Notch2, CD175s, CD82, CD49b, CD95, CD63, CD245, CD58, CD108, B2-microglobulin, CD155, CD298, CD44, CD49c, CD105, CD166, CD230, HLA-ABC, CD13, CD29, CD49e, CD59, CD73, CD81, CD90, CD98, CD147, CD151 and CD276. The IMP cells may express detectable levels of any number and combination of these markers. The IMP cells preferably express detectable levels of all of these markers.

The IMP cells preferably express detectable levels of one or more of CD72, CD133, CD192, CD207, CD144, CD41b, FMC7, CD75, CD3e, CD37, CD158a, CD172b, CD282, CD100, CD94, CD39, CD66b, CD158b, CD40, CD35, CD15, PAC-1, CLIP, CD48, CD278, CD5, CD103, CD209, CD3, CD197, HLA-DM, CD20, CD74, CD87, CD129, CDw329, CD57, CD163, TPBG, CD206, CD243 (BD), CD19, CD8, CD52, CD184, CD107b, CD138, CD7, CD50, HLA-DR, CD158e2, CD64, DCIR, CD45, CLA, CD38, CD45RB, CD34, CD101, CD2, CD41a, CD69, CD136, CD62P, TCR alpha beta, CD16b, CD1a, ITGB7, CD154, CD70, CDw218a, CD137, CD43, CD27, CD62L, CD30, CD36, CD150, CD66, CD212, CD177, CD142, CD167, CD352, CD42a, CD336, CD244, CD23, CD45RO, CD229, CD200, CD22, CDH6, CD28, CD18, CD21, CD335, CD131, CD32, CD157, CD165, CD107a, CD1b, CD332, CD180, CD65 and CD24. The IMP cells may express detectable levels of any number and combination of these markers. The IMP cells preferably express detectable levels of all of these markers.

The one or more IMP cells may be part of any of the populations disclosed in GB 1410504.3.

The IMP cells are preferably capable of adhering to a specific, damaged tissue in a patient. Adherence and adhesion assay are known in the art (Humphries, Methods Mol Biol. 2009; 522:203-10).

The IMP cells are preferably capable of proliferating in a specific, damaged tissue in a patient. Cell proliferation assays are well known in the art. Such assays are commercially available, for instance from Life Technologies®.

The IMP cells are preferably capable of promoting angiogenesis in a specific, damaged tissue in a patient. Angiogenesis assays are known in the art (Auerback et al., Clin Chem. 2003 January; 49(1):32-40).

The IMP cells are preferably capable of having anti-inflammatory effects in a damaged tissue of a patient. The ability of the IMP cells of the invention to have anti-inflammatory effects may also be measured using standard assays known in the art. Suitable methods include, but are not limited to, enzyme-linked immunosorbent assays (ELISAs) for the secretion of cytokines, enhanced mixed leukocyte reactions and up-regulation of co-stimulatory molecules and maturation markers, measured by flow cytometry. The cytokines measured are typically interleukins, such as interleukin-8 (IL-8), selectins, adhesion molecules, such as Intercellular Adhesion Molecule-1 (ICAM-1), and chemoattractant proteins, such as monocyte chemotactic protein-1 (MCP-1) and tumour necrosis factor alpha (TNF-alpha). Assays for these cytokines are commercially-available. Anti-inflammatory factors are preferably detected and measured using the Luminex® assay. Such assays are commercially available from Life Technologies®.

The IMP cells preferably secrete detectable levels of one or more of interleukin-6 (IL-6), IL-8, C-X-C motif chemokine 10 (CXCL10; interferon gamma-induced protein 10; IP-10), Chemokine (C-C motif) ligand 2 (CCL2; monocyte chemotactic protein-1; MCP-1) and Chemokine (C-C motif) ligand 5 (CCL5; regulated on activation, normal T cell expressed and secreted; RANTES). The IMP cells may secrete any number and combination of these factors. The IMP cells preferably secrete all of these markers.

The IMP cells preferably secrete an increased amount of one or more of IL-6, IL-8, IP-10, MCP-1 and RANTES compared with a MSC. The IMP cells may secrete an increased amount of any number and combination of these factors. The IMP cells preferably secrete an increased amount of all of these markers.

The IMP cells preferably secrete a decreased amount of interleukin-10 (IL-10) and/or IL-12 compared with a mesenchymal stem cell MSC. IL-10 and IL-12 are pro-inflammatory cytokines.

The IMP cells will express a variety of different other markers over and above those discussed above. Some of these will assist the IMP cells will their ability to have anti-inflammatory effects in a damaged tissue. Any of the IMP cells of the invention may further express detectable levels of one or more of (i) insulin-like growth factor-1 (IGF-1), (ii) IGF-1 receptor; (iii) C-C chemokine receptor type 1 (CCR1), (iv) stromal cell-derived factor-1 (SDF-1), (v) hypoxia-inducible factor-1 alpha (HIF-1 alpha), (vi) Akt1 and (vii) hepatocyte growth factor (HGF) and/or granulocyte colony-stimulating factor (G-CSF).

IGF-1 receptors promote migration capacity towards an IGF-1 gradient. One of the mechanisms by which IGF-1 increases migration is by up-regulating CXCR4 on the surface of the cells, which makes them more sensitive to SDF-1 signaling.

CCR1 is the receptor for CCL7 (previously known as MCP3) increases homing and engraftment capacity of MSCs (and so would be expected to have the same effect for the IMP cells of the invention) and can increase the capillary density in injured myocardium through paracrine signalling.

HIF-1 alpha activates pathways that increase oxygen delivery and promote adaptive pro-survival responses. Among the many target genes of HIF-1 alpha are erythropoietin (EPO), endothelin and VEGF (with its receptor Flk-1). IMP cells that express or express an increased amount of HIF-1 alpha will have upregulated expression of paracrine stimuli of for example several vasculogenic growth factors that may promote a more therapeutic subtype. As described in more detail below, the IMP cells of the invention can be preconditioned into a more therapeutic subtype by culturing them under hypoxic conditions (less than 20% oxygen), such as for example about 2% or about 0% oxygen.

Akt1 is an intracellular serine/threonine protein kinase that plays a key role in multiple cellular processes such as glucose metabolism, cell proliferation, apoptosis, transcription and cell migration. Overexpression of Akt1 has been shown to prevent rat MSCs from undergoing apoptosis and will have the same effect in the IMP cells of the invention. Protection from apoptosis will enhance the therapeutic effect of the IMP cells.

The overexpression of HGF by MSCs has been shown to prevent post-ischemic heart failure by inhibition of apoptosis via calcineurin-mediated pathway and angiogenesis. HGF and G-CSF exhibit synergistic effects in this regard. MSCs that have a high expression of HGF and its receptor c-met also have an increased migratory capacity into the damaged tissue, achieved through hormonal, paracrine and autocrine signaling. The same will be true for the IMP cells of the invention expressing HGF and/or G-CSF.

The IMP cells may express detectable levels of one or more of (i) to (vii) defined above. The IMP cells of the invention preferably express an increased amount of one or more of (i) to (vii) compared with MSCs. Quantitative assays for cell markers are described above. The detectable expression of these markers and their level of expression may be measured as discussed above.

Any of the IMP cells may express detectable levels of one or more of (i) vascular endothelial growth factor (VEGF), (ii) transforming growth factor beta (TGF-beta), (iii) insulin-like growth factor-1 (IGF-1), (iv) fibroblast growth factor (FGF), (v) tumour necrosis factor alpha (TNF-alpha), (vi) interferon gamma (IFN-gamma) and (vii) interleukin-1 alpha (IL-1 alpha). Conditioned medium from cells overexpressing VEGF has been shown to alleviate heart failure in a hamster model. Hence, the IMP cells of the invention which express or express an increased amount of VEGF will have the same effect of damaged cardiac tissue.

The IMP cells may express detectable levels of one or more of (i) to (vii). The IMP cells may express an increased amount of one or more of (i) to (vii) compared with MSCs. Quantitative assays for cell markers are described above. The detectable expression of these markers and their level of expression may be measured as discussed above.

In both sets of definitions of (i) to (vii) given above, any combination of one or more of (i) to (vii) may be expressed or expressed in an increased amount. For instance, for each definition of (i) to (vii), the IMP cells may express detectable levels of, or express an increased amount of, (i); (ii); (ii); (iii); (iv); (v); (vi); (vii); (i) and (ii); (i) and (iii); (i) and (iv); (i) and (v); (i) and (vi); (i) and (vii); (ii) and (iii); (ii) and (iv); (ii) and (v); (ii) and (vi); (ii) and (vii); (iii) and (iv); (iii) and (v); (iii) and (vi); (iii) and (vii); (iv) and (v); (iv) and (vi); (iv) and (vii); (v) and (vi); (v) and (vii); (vi) and (vii); (i), (ii) and (iii); (i), (ii) and (iv); (i), (ii) and (v); (i), (ii) and (vi); (i), (ii) and (vii); (i,), (iii) and (iv); (i), (iii) and (v); (i), (iii) and (vi); (i), (iii) and (vii); (i), (iv) and (v); (i), (iv) and (vi); (i), (iv) and (vii); (i), (v) and (vi); (i), (v) and (vii); (i), (vi) and (vii); (ii), (iii) and (iv); (ii), (iii) and (v); (ii), (iii) and (vi); (ii), (iii) and (vii); (ii), (iv) and (v); (ii), (iv) and (vi); (ii), (iv) and (vii); (ii), (v) and (vi); (ii), (v) and (vii); (ii), (vi) and (vii); (iii), (iv) and (v); (iii), (iv) and (vi); (iii), (iv) and (vii); (iii), (v) and (vi); (iii), (v) and (vii); (iii), (vi) and (vii); (iv), (v) and (vi); (iv), (v) and (vii); (iv), (vi) and (vii); (v), (vi) and (vii); (i), (ii), (iii) and (iv); (i), (ii), (iii) and (v); (i), (ii), (iii) and (vi); (i), (ii), (iii) and (vii); (i), (ii), (iv) and (v); (i), (ii), (iv) and (vi); (i), (ii), (iv) and (vii); (i), (ii), (v) and (vi); (i), (ii), (v) and (vii); (i), (ii), (vi) and (vii); (i), (iii), (iv) and (v); (i), (iii), (iv) and (vi); (i), (iii), (iv) and (vii); (i), (iii), (v) and (vi); (i), (iii), (v) and (vii); (i), (iii), (vi) and (vii); (i), (iv), (v) and (vi); (i), (iv), (v) and (vii); (i), (iv), (vi) and (vii); (i), (v), (vi) and (vii); (ii), (iii), (iv) and (v); (ii), (iii), (iv) and (vi); (ii), (iii), (iv) and (vii); (ii), (iii), (v) and (vi); (ii), (iii), (v) and (vii); (ii), (iii), (vi) and (vii); (ii), (iv), (v) and (vi); (ii), (iv), (v) and (vii); (ii), (iv), (vi) and (vii); (ii), (v), (vi) and (vii); (iii), (iv), (v) and (vi); (iii), (iv), (v) and (vii); (iii), (iv), (vi) and (vii); (iii), (v), (vi) and (vii); (iv), (v), (vi) and (vii); (i), (ii), (iii), (iv) and (v); (i), (ii), (iii), (iv) and (vi); (i), (ii), (iii), (iv) and (vii); (i), (ii), (iii), (v) and (vi); (i), (ii), (iii), (v) and (vii); (i), (ii), (iii), (vi) and (vii); (i), (ii), (iv), (v) and (vi); (i), (ii), (iv), (v) and (vii); (i), (ii), (iv), (vi) and (vii); (i), (ii), (v), (vi) and (vii); (i), (iii), (iv), (v) and (vi); (i), (iii), (iv), (v) and (vii); (i), (iii), (iv), (vi) and vii); (i), (iii), (v), (vi) and (vii); (i), (iv), (v), (vi) and (vii); (ii), (iii), (iv), (v) and (vi); (ii), iii), (iv), (v) and (vii); (ii), (iii), (iv), (vi) and (vii); (ii), (iii), (v), (vi) and (vii); (ii), (iv), (v), (vi) and (vii); (iii), (iv), (v), (vi) and vii); (i), (ii), (iii), (iv), (v) and (vi); (i), (ii), (iii), (iv), (v) and (vii); (i), (ii), (iii), (iv), (vi) and (vii); (i), (ii), (iii), (v), (vi) and (vii); (i), (ii), (iv), (v), (vi) and (vii); (i), (iii), (iv), (v), (vi) and (vii); (ii), (iii), (iv), (v), (vi) and (vii); or (i), (ii), (iii), (iv), (v), (vi) and (vii). The combinations for each definition of (i) to (vii) are independently selectable from this list.

In addition to any of the markers discussed above, the IMP cells preferably also express detectable levels of, LIF and/or PDGF receptors. The IMP cells of the invention preferably express an increased amount of LIF and/or PDGF receptors compared with mesenchymal stem cells. The PDGF receptors are preferably PDGF-A receptors and/or PDGF-B receptors. MSCs that have high expression of these receptors can migrate effectively into areas in which platelets have been activated, such as wounds and thrombotic vessels. The same will be true of IMP cells expressing or expressing an increased amount of the receptors.

The PMLs and/or the IMP cells are preferably autologous. In other words, the cells are preferably derived from the patient into which the cells will be administered. Alternatively, the PMLs and/or IMP cells are preferably allogeneic. In other words, the cells are preferably derived from a patient that is immunologically compatible with the patient into which the cells will be administered.

PMLs and/or IMP cells may be isolated using a variety of techniques including antibody-based techniques. Cells may be isolated using negative and positive selection techniques based on the binding of monoclonal antibodies to those surface markers which are present on the PMLs and/or IMP cells (see above). Hence, the PMLs and/or IMP cells may be separated using any antibody-based technique, including fluorescent activated cell sorting (FACS) and magnetic bead separation.

As discussed below, the PMLs and/or IMP cells may be treated ex vivo. Thus the cells may be loaded or transfected with a therapeutic or diagnostic agent and then used therapeutically in the methods of the invention.

The one or more PMLs and/or IMP cells may be produced using any known method. The one or more PMLs are preferably produced using the method described in International Application No. PCT/GB2012/051600 (published as WO 2013/005053). The one or more IMP cells are preferably produced using the method disclosed in GB 1410504.3.

This typically involves culturing MCs under conditions which induce the MCs to differentiate into PMLs or IMP cells and then harvesting and culturing the PMLs or IMP cells which express the markers discussed above.

Mononuclear cells (MCs) and methods of isolating them are known in the art. The MCs may be primary MCs isolated from bone marrow. The MCs are preferably peripheral blood MCs (PBMCs), such as lymphocytes, monocytes and/or macrophages. PBMCs can be isolated from blood using a hydrophilic polysaccharide, such as Ficoll®. For instance, PBMCs may be isolated from blood using Ficoll-Paque® (a commercially-available density medium).

Before they are cultured, the MCs may be exposed to a mesenchymal stem cell enrichment cocktail. The cocktail preferably comprises antibodies that recognise CD3, CD14, CD19, CD38, CD66b (which are present on unwanted cells) and a component of red blood cells. Such a cocktail cross links unwanted cells with red blood cells forming immunorosettes which may be removed from the wanted MCs. A preferred cocktail is RosetteSep®.

Conditions suitable for inducing MCs to differentiate into mesenchymal cells (tissue mainly derived from the mesoderm) are known in the art. For instance, suitable conditions are disclosed in Capelli, C., et al. (Human platelet lysate allows expansion and clinical grade production of mesenchymal stromal cells from small samples of bone marrow aspirates or marrow filter washouts. Bone Marrow Transplantation, 2007. 40: p. 785-791). These conditions may also be used to induce MCs to differentiate into PMLs in accordance with the invention.

The method preferably comprises culturing MCs with plasma lysate to induce the MCs to differentiate into PMLs or IMP cells. Platelet lysate refers to the combination of natural growth factors contained in platelets that has been released through lysing those platelets. Lysis can be accomplished through chemical means (i.e. CaCl₂), osmotic means (use of distilled H₂O) or through freezing/thawing procedures. Platelet lysate can be derived from whole blood as described in U.S. Pat. No. 5,198,357. Platelet lysate is preferably prepared as described in PCT/GB12/052911 (published as WO 2013/076507). The plasma lysate is preferably human plasma lysate.

For instance, the MCs may be cultured in a medium comprising platelet lysate for sufficient time to induce the MCs to differentiate into PMLs or IMP cells. The sufficient time is typically from about 15 to about 25 days, preferably about 22 days. The medium preferably comprises about 20% or less platelet lysate by volume, such as about 15% or less by volume or about 10% or less by volume. The medium preferably comprises from about 5% to about 20% of platelet lysate by volume, such as from about 10% to about 15% by volume. The medium preferably comprises about 10% of platelet lysate by volume.

Alternatively, the MCs may be exposed to a mesenchymal enrichment cocktail and then cultured in a medium comprising platelet lysate for sufficient time to induce the MCs to differentiate into PMLs or IMP cells. The sufficient time is typically from about 15 to about 25 days, preferably about 22 days.

The medium is preferably Minimum Essential Medium (MEM). MEM is commercially available from various sources including Sigma-Aldrich. The medium preferably further comprises one or more of heparin, L-glutamine and penicillin/streptavidin (P/S). The L-glutamine may be replaced with GlutaMAX® (which is commercially-available from Life Technologies).

The MCs are typically cultured under conditions which allow the PMLs or IMP cells to adhere. Suitable conditions are discussed in more detail above.

The MCs are preferably cultured under low oxygen conditions. The MCs are preferably cultured at less than about 20% oxygen (O₂), such as less than about 19%, less than about 18%, less than about 17%, less than about 16%, less than about 15%, less than about 14%, less than about 13%, less than about 12%, less than about 11%, less than about 10%, less than about 9%, less than about 8%, less than about 7%, less than about 6%, less than about 5%, less than about 4%, less than about 3%, less than about 2% or less than about 1% oxygen (O₂). The MCs are preferably cultured at from about 0% to about 19% O₂, such as from about 1% to about 15% O₂, from about 2% to about 10% O₂ or from about 5% to about 8% O₂. The MCs are most preferably cultured at about 0% O₂. The figures for % oxygen (or % O₂) quoted above relate to % by volume of oxygen in the gas supplied to the cells during culture, for instance by the cell incubator. It is possible that some oxygen may leak into the incubator or enter when the door is opened.

The MCs are most preferably cultured in the presence of platelet lysate and under low oxygen conditions. This combination mimics the natural conditions in the damaged tissue and so result in healthier and more therapeutically potent cells. Conventional cell culture is performed in 20% or 21% oxygen (approximately the atmospheric content) but there is no place in the human body that has this oxygen level. The epithelial cells in the lungs would “see” this oxygen level, but once the oxygen is dissolved and leaves the lungs, it decreases to around 17%. From there, it decreases even further to about 1-2% in the majority of the tissues, but being as low as 0.1% in avascular tissues such as the cartilage in the joints.

Producing one or more PMLs or IMP cells also comprises harvesting and culturing PMLs or IMP cells which have the necessary marker expression pattern as discussed above. The PMLs or IMP cells having the necessary marker expression pattern may be harvested using any antibody-based technique, including fluorescent activated cell sorting (FACS) and magnetic bead separation. FACS is preferred.

As will be clear from the discussion above, the production of one or more PMLs or IMP cells is carried out in clinically relevant conditions, i.e. in the absence of trace amounts of endotoxins and other environmental contaminants, such as lipopolysaccharides, lipopeptides and peptidoglycans, etc. This makes the PMLs or IMP cells particularly suitable for administration to patients.

The MCs are preferably obtained from a patient or an allogeneic donor.

The one or more therapeutic cells are preferably one or more mesenchymal stem cells (MSCs). Suitable MSCs are known in the art. Any of the MSCs disclosed above may be used.

The one or more therapeutic cells are preferably one or more mesenchymal precursor cells (MPCs). The one or more therapeutic cells are more preferably one or more human MPCs.

The one or more therapeutic cells are preferably one or more dendritic cells.

The one or more therapeutic cells are preferably one or more platelets.

The one or more therapeutic cells are preferably one or more fibroblasts.

The one or more therapeutic cells are preferably one or more myofibroblasts.

The one or more therapeutic cells are preferably human. The one or more therapeutic cells may be derived from any of the animals or mammals discussed above with reference to the source of the PML and IMP cells.

The one or more therapeutic cells are typically cultured in vitro before being combined with the other components in the composition. Techniques for culturing cells are well known to a person skilled in the art. The cells are may be cultured under standard conditions of 37° C., 5% CO₂ in medium without serum. The cells are preferably cultured under low oxygen conditions as discussed in more detail below. The cells may be cultured in any suitable flask or vessel, including wells of a flat plate such as a standard 6 well plate. Such plates are commercially available from Fisher scientific, VWR suppliers, Nunc, Starstedt or Falcon. The wells typically have a capacity of from about 1 ml to about 4 ml.

The flask, vessel or wells within which the population is contained or cultured may be modified to facilitate handling of the cells. For instance, the flask, vessel or wells may be modified to facilitate culture of the cells, for instance by including a growth matrix. The flask, vessel or wells may be modified to allow attachment of the cells or to allow immobilization of the cells onto a surface. One or more surfaces may be coated with extracellular matrix proteins such as laminin or collagen or any other capture molecules that bind to the cells and immobilize or capture them on the surface(s).

The cells may be modified ex vivo using any of the techniques described herein. For instance, the population may be transfected or loaded with therapeutic or diagnostics agents. The population may then be used in the methods of treatment discussed in more detail below.

### Medicaments, Methods and Therapeutic Use

A composition of the invention may be used in a method of therapy of the human or animal body. Thus, the invention provides a composition of the invention for use in a method of repairing damaged tissue in a patient, the method comprising administering to the patient a therapeutically effective amount of the composition.

The invention also provides a composition of the invention for use in a method of inhibiting senescence in a patient in need thereof, the method comprising administering to the patient a therapeutically effective amount of the composition. Senescence is the gradual deterioration of function of cells and the organism comprising those cells. The invention may therefore concern inhibiting senescence in the patient. The invention may therefore concern inhibiting senescence in one or more tissues of the patient. The one or more tissues may be any of those discussed below. The composition of the invention typically increases the Hayflick limit of a cell population in vitro. The Hayflick limit is the number of times a normal cell population (typically a normal human cell population) will divide until cell division stops and is discussed in Shay and Wright, Nat Rev Mol Cell Biol. 2000 October; 1(1):72-6. The invention also concerns inhibiting ageing.

The invention further provides a composition of the invention for use in a method of promoting hair growth in a patient, the method comprising administering to the patient a therapeutically effective amount of the composition.

The invention also provides a method of repairing damaged tissue in a patient, comprising administering to the patient a therapeutically effective amount of a composition of the invention, and thereby treating the damaged tissue in the patient. The damaged tissue is preferably damaged by injury or disease. In addition, the invention provides a method of inhibiting senescence in a patient, comprising administering to the patient a therapeutically effective amount of a composition of the invention, and thereby inhibiting the senescence in the patient. The invention also provides a method of promoting hair growth in a patient, comprising administering to the patient a therapeutically effective amount of a composition of the invention.

The damaged or senescent tissue is preferably derived from mesoderm. The damaged or senescent tissue is more preferably tendon, ligament, cardiac, bone, cartilage, liver, kidney, lung tissue or vaginal tissue. The method may therefore be used to treat tendon, ligament, cardiac, bone, cartilage, liver, kidney, lung tissue or vaginal tissue disease or injury in the patient. The method may also be used to inhibit tendon, ligament, cardiac, bone, cartilage, liver, kidney, lung tissue or vaginal tissue senescence in the patient.

In addition, the method may be for treating vaginal atrophy. Vaginal atrophy (also known as atrophic vaginitis or urogenital atrophy) is an inflammation of the vagina (and the outer urinary tract) due to the thinning and shrinking of the tissues, as well as decreased lubrication. It is typically caused by a decrease in secretion of the hormone estrogen.

The cardiac injury, disease or senescence is preferably selected from myocardial infarct (MI), left ventricular hypertrophy, right ventricular hypertrophy, emboli, heart failure, congenital heart deficit, heart valve disease, arrhythmia and myocarditis.

MI increases the levels of VEGF and EPO released by the myocardium. Furthermore, MI is associated with an inflammatory reaction and infarcted tissue also releases macrophage migration inhibitory factor (MIF), interleukin (IL-6) and KC/Gro-alpha. CCL7 (previously known as MCP3), CXCL1, CXCL2 are significantly upregulated in the heart following myocardial infarct (MI) and might be implicated in regulating engraftment and homing of MSCs to infarcted myocardium.

In a myocardial infarct mice model, IL-8 was shown to highly up-regulate gene expression primarily in the first 2 days post-MI. Remarkably, the increased IL-8 expression was located predominantly in the infarcted area and the border zone, and only to a far lesser degree in the spared myocardium. By activating CXCR2, MIF displays chemokine-like functions and acts as a major regulator of inflammatory cell recruitment and atherogenesis.

The bone disease or injury is preferably selected from fracture, Salter-Harris fracture, greenstick fracture, bone spur, craniosynostosis, Coffin-Lowry syndrome, fibrodysplasia ossificans progressive, fibrous dysplasia, Fong Disease (or Nail-patella syndrome), hypophosphatasia, Klippel-Feil syndrome, Metabolic Bone Disease, Nail-patella syndrome, osteoarthritis, osteitis deformans (or Paget's disease of bone), osteitis fibrosa cystica (or Osteitis fibrosa or Von Recklinghausen's disease of bone), osteitis pubis, condensing osteitis (or osteitis condensans), osteitis condensans ilii, osteochondritis dissecans, osteogenesis imperfecta, osteomalacia, osteomyelitis, osteopenia, osteopetrosis, osteoporosis, osteonecrosis, porotic hyperostosis, primary hyperparathyroidism, renal osteodystrophy, bone cancer, a bone lesion associated with metastatic cancer, Gorham Stout disease, primary hyperparathyroidism, periodontal disease, and aseptic loosening of joint replacements. The bone cancer can be Ewing sarcoma, multiple myeloma, osteosarcoma (giant tumour of the bone), osteochondroma or osteoclastoma. The metastatic cancer that results in a bone lesion can be breast cancer, prostate cancer, kidney cancer, lung cancer and/or adult T-cell leukemia.

Administration of a composition of the invention in accordance with a method of the invention preferably results in improved tissue regeneration. Furthermore, administration of a composition of the invention in accordance with a method of the invention preferably results in reduced apoptosis.

The invention concerns administering to the patient a therapeutically effective amount of a composition of the invention. A therapeutically effective amount is an amount which ameliorates one or more symptoms of the damaged or senescent tissue, or promotes hair growth. A therapeutically effective amount is preferably an amount which repairs the damaged or senescent tissue, or results in hair growth.

The composition of the invention of the invention may be administered to any suitable patient. The patient is generally human. The patient may be any mammal. Such mammals include commercially farmed animals, such as a horses, cattle, sheep or pigs, laboratory animals, such as mice or rats, and pets, such as cats, dogs, rabbits or guinea pigs.

The patient may be an infant, a juvenile or an adult. The patient may be known to have a damaged or senescent tissue or is suspected of having a damaged or senescent tissue. The patient may be susceptible to, or at risk from, the relevant disease, injury or senescence. For instance, the patient may be genetically predisposed to heart failure.

The invention may be used in combination with other means of, and substances for, repairing damaged tissue, inhibiting senescence or providing pain relief. In some cases, the composition of the invention may be administered simultaneously, sequentially or separately with other substances which are intended for repairing the damaged tissue, inhibiting senescence or providing pain relief. The composition of the invention may be used in combination with existing substances for repairing damaged tissue or inhibiting senescence and may, for example, be simply mixed with such treatments. Thus the invention may be used to increase the efficacy of existing substances for damaged tissue or inhibiting senescence. Similarly, the composition of the invention may be used in combination with other means of, and substances for, promoting hair growth.

As set out above, the composition of the invention may comprise one or more therapeutic cells. In all instances, the one or more therapeutic cells are preferably derived from the patient or an allogeneic donor. Deriving the cells from the patient should ensure that the cells are themselves not rejected by the patient's immune system. Any difference between the donor and recipient will ultimately cause clearance of the cells, but not before they have repaired at least a part of the damaged or senescent tissue.

One aspect of the invention concerns administering to the patient a therapeutically effective number of therapeutic cells to the patient. A therapeutically effective number is a number which ameliorates one or more symptoms of the damage, disease, injury or senescence. A therapeutically effective number is preferably a number which repairs the damaged tissue or treats the disease, injury or senescence.

The therapeutic cells may be loaded or transfected with a therapeutic and/or diagnostic agent. A therapeutic agent may help to repair the damaged or senescent tissue. A diagnostic agent, such as a fluorescent molecule, may help to identify the location of the composition in the patient. The cells may be loaded or transfected using any method known in the art. The loading of cells may be performed in vitro or ex vivo. In each case, the cells may simply be in contact with the agent in culture. Alternatively, the cells may be loaded with an agent using delivery vehicle, such as liposomes. Such vehicles are known in the art.

The transfection of cells, such as PMLs or IMP cells, may be performed in vitro or ex vivo. Alternatively, stable transfection may be performed at the MC stage allowing PMLs expressing the transgene to be differentiated from them. The cells are typically transfected with a nucleic acid encoding the agent. For instance, viral particles or other vectors encoding the agent may be employed. Methods for doing this are known in the art.

The nucleic acid gives rise to expression of the agent in the cells. The nucleic acid molecule will preferably comprise a promoter which is operably linked to the sequences encoding the agent and which is active in the PMLs or which can be induced in the cells.

In a particularly preferred embodiment, the nucleic acid encoding the agent may be delivered via a viral particle. The viral particle may comprise a targeting molecule to ensure efficient transfection. The targeting molecule will typically be provided wholly or partly on the surface of the virus in order for the molecule to be able to target the virus to the cells.

Any suitable virus may be used in such embodiments. The virus may, for example, be a retrovirus, a lentivirus, an adenovirus, an adeno-associated virus, a vaccinia virus or a herpes simplex virus. In a particularly preferred embodiment the virus may be a lentivirus. The lentivirus may be a modified HIV virus suitable for use in delivering genes. The lentivirus may be a SIV, FIV, or equine infectious anemia virus (EQIA) based vector. The virus may be a moloney murine leukaemia virus (MMLV). The viruses used in the invention are preferably replication deficient.

Viral particles do not have to be used. Any vector capable of transfecting the cells may be used, such as conventional plasmid DNA or RNA transfection.

Uptake of nucleic acid constructs may be enhanced by several known transfection techniques, for example those including the use of transfection agents. Examples of these agents includes cationic agents, for example, calcium phosphate and DEAE-Dextran and lipofectants, for example, lipofectAmine, fugene and transfectam.

The cells may be loaded or transfected under suitable conditions. The cells and agent or vector may, for example, be contacted for between five minutes and ten days, preferably from an hour to five days, more preferably from five hours to two days and even more preferably from twelve hours to one day.

In some embodiments, MCs may be recovered from a patient, converted into PMLs and/or IMP cells as discussed above, loaded or transfected in vitro and then returned to the same patient. In such instances, the PMLs and/or IMP cells employed in the invention, will be autologous cells and fully matched with the patient. In a preferred case, the cells employed in the invention are recovered from a patient and utilised ex vivo and subsequently returned to the same patient.

### Pharmaceutical Compositions and Administration

The composition of the invention may be formulated using any suitable method. Formulation of cells with standard pharmaceutically acceptable carriers and/or excipients may be carried out using routine methods in the pharmaceutical art. The exact nature of a formulation will depend upon several factors including the cells to be administered and the desired route of administration. Suitable types of formulation are fully described in Remington's Pharmaceutical Sciences, 19^{th} Edition, Mack Publishing Company, Eastern Pennsylvania, USA.

The composition of the invention is typically sterile.

The composition of the invention may be administered by any route. Suitable routes include, but are not limited to, topical, subcutaneous, intravenous, intramuscular, intraperitoneal or other appropriate administration routes. The composition is preferably administered directly to the damaged or senescent tissue. The composition may also be administered topically to promote hair growth in the region to which the composition is administered. Vaginal atrophy may be treated by topically applying a composition of the invention.

The composition of the invention may be prepared together with a physiologically acceptable carrier or diluent. Suitable carriers or excipients are, for example, water, saline, dextrose, glycerol, of the like and combinations thereof. Lyophilised compositions are typically rehydrated before therapeutic use.

In addition, if desired, the pharmaceutical compositions of the invention may contain minor amounts of auxiliary substances such as wetting or emulsifying agents, pH buffering agents, and/or adjuvants which enhance effectiveness. Such agents are known in the art. The composition preferably comprises human serum albumin. The composition is preferably xeno-free.

One suitable carrier or diluent is Plasma-Lyte A®. This is a sterile, nonpyrogenic isotonic solution for intravenous administration. Each 100 mL contains 526 mg of Sodium Chloride, USP (NaCl); 502 mg of Sodium Gluconate (C6H11NaO7); 368 mg of Sodium Acetate Trihydrate, USP (C2H3NaO2.3H2O); 37 mg of Potassium Chloride, USP (KCl); and 30 mg of Magnesium Chloride, USP (MgCl2.6H2O). It contains no antimicrobial agents. The pH is adjusted with sodium hydroxide. The pH is 7.4 (6.5 to 8.0).

The composition of the invention is administered in a manner compatible with the dosage formulation and in such amount will be therapeutically effective. The quantity to be administered depends on the subject to be treated, capacity of the subject's immune system and the degree of repair or treatment desired. Precise amounts required to be administered may depend on the judgment of the practitioner and may be peculiar to each subject.

Any suitable amount of the composition of the invention may be administered to the subject. For instance, the amount of the pharmaceutical composition of the invention which is administered may range from about 0.1 g to 100 g, such as from about 0.5 g to about 75 g, from about 1 g to about 50 g, from about 2 g to about 20 g or from about 3 g to 10 g.

Where the composition contains therapeutic cells, the quantity of the therapeutic cells to be administered depends on the subject to be treated, capacity of the subject's immune system and the degree of repair desired. Precise amounts of cells required to be administered may depend on the judgment of the practitioner and may be peculiar to each subject.

Any suitable number of cells may be administered to a subject. For example, at least, or about, 0.2×10⁶, 0.25×10⁶, 0.5×10⁶, 1.5×10⁶, 4.0×10⁶ or 5.0×10⁶ cells per kg of patient may administered. For example, at least, or about, 10⁵, 10⁶, 10⁷, 10⁸, 10⁹ cells may be administered. As a guide, the number of cells to be administered may be from 10⁵ to 10⁹, preferably from 10⁶ to 10⁸. Typically, up to 2×10⁸ IMP cells are administered to each patient. Any of the specific numbers discussed above may be administered.

The composition of the invention may be in lyophilised form. The lyophilisation process would produce a stabilized, freeze-dried pharmaceutical powder comprising the growth factors and, potentially, the other bioactive components described above. This lyophilised composition could be combined with other technologies for effective treatment of patients with damaged or senescent tissue, or patients in need of promotion of hair growth, with much greater long term stability at different temperatures. This would circumvent the logistical process of preparation and transfer of the treatment.

One embodiment is a dry, lyophilised composition of the invention. When combined with water, just prior to treatment, a gel-like consistency would be formed.

In other embodiments, a pharmaceutical composition of the invention or a platelet lysate of the invention could alternatively be combined with different:
formulation/delivery methods, such as:
   Lotion, Shake lotion, Cream, Ointment, Gel, Foam, Transdermal patch, Powder, Solid, Sponge, Tape, Paste, bandage, gauze, syringe, spray
treatments, such as:
   Mesenchymal stem cells, hematopoietic stem cells, mononuclear cells, endothelial progenitor cells, mesodermal progenitor cells, antibiotics, analgesics, silver, debriding agents, medical devices
Methods of packaging, such as:
   Sterile package, bottle, box, can
Methods of storage
   Ideally, room temperature powder; refrigerated or frozen, as necessary.

### Methods of Producing a Population of Cells

The invention provides a method of producing a population of cells for use in a method of treating damaged tissue or inhibiting senescence, or promoting hair growth. The method involves culturing MCs, PMLs and/or IMPs in the presence of the composition of the invention, and allowing at least some of the MCs, PMLs and/or IMPs to proliferate.

The MCs and methods of isolating them are known in the art. The MCs may be primary MCs isolated from bone marrow. The MCs are preferably peripheral blood MCs (PBMCs), such as lymphocytes, monocytes and/or macrophages. PBMCs can be isolated from blood using a hydrophilic polysaccharide, such as Ficoll®. For instance, PBMCs may be isolated from blood using Ficoll-Paque® (a commercially-available density medium).

The IMPs and/or PMLs are preferably as defined above.

The method of the invention may be carried out in clinically relevant conditions, i.e. in the absence of trace amounts of endotoxins and other environmental contaminants, such as lipopolysaccharides, lipopeptides and peptidoglycans, etc. This makes the resultant population of cells particularly suitable for administration to patients.

The MCs, PMLs or IMPs are preferably derived from a patient or an allogeneic donor. In this instance, the population of cells produced by the method of the invention will be autologous with the patient and therefore will not be rejected upon administration to the patient.

The MCs, PMLs or IMPs may also be derived from one or more different patients that are immunologically compatible with the patient into which the cells will ultimately be administered. In this instance, the population of cells produced by the method of the invention will be allogeneic or semi-allogeneic with the patient. The chance of rejection upon administration of the population to the patient will therefore be reduced.

The invention also provides a population of cells that is suitable for administration to a patient and is produced using the method of the invention. The population of cells may comprise PMLs, MSCs or IMPs, as defined above.

The following Examples illustrate the invention.

### Example 1—Preparation of Composition

A composition of the invention was prepared from human platelets. Platelets for human transfusion have a short shelf life and so are readily available from the Welsh Blood Service.

Platelets were placed in cryopreservation bags, and bags transferred to liquid nitrogen until the contents were frozen completely. Bags were then transferred to a 37° C. water bath until the contents were completely thawed. This freeze-thaw cycle was repeated four times, resulting in the lysis of the platelets and release of their contents into the plasma phase. The lysate was transferred to centrifuge tubes and centrifuged at 3200 g for 20 minutes at room temperature to pellet the platelet debris. The supernatant was collected into fresh tubes, poring through a fine (40 μm) mesh to reduce the debris. The composition was then stored in aliquots at −80° C.

### Example 2—Preparation of a Therapeutic Gel

A therapeutic gel was made by mixing the composition of Example 1 with methylcellulose in PBS. In this instance, the final gel was 2.5% Methylcellulose, 0.5×PBS and 50% composition. However, a similar method can be used to prepare any gel formulation.

The composition produced in Example 1 was thawed overnight at 4° C. and centrifuged at 3200 g for 20 minutes at room temperature. The supernatant was collected into fresh tubes, poring through a fine (40 μm) mesh to reduce debris.

The required quantity of methylcellulose was weighed onto and packaged into parchment coated foil, and carefully sealed. The required quantity of PBS was measured into a bottle and a stirrer bar added. The packaged methylcellulose and the bottle of PBS were then autoclaved.

Following autoclaving the PBS was warmed in a 80° C. water bath. In a safety cabinet, the methylcellulose was unwrapped and dispensed into the hot PBS, stirring briskly. The methylcellulose suspension was left to stir and cool to hand hot.

The thawed composition was then added to the hand hot methylcellulose suspension. The mixture was stirred for a further 20 minutes. Finally, the resultant gel was transferred to the fridge, where complete hydration of the methylcellulose resulted in thickening and clarification of the gel.

### Example 3—Optimisation of Gel Formulation

A gel formulation comprising the composition and methylcellulose or PF-127 was optimised by investigating the gelling temperature, viscosity of gel, its ability to enhance fibroblast proliferation and its suitability for long term storage.

### Methods

### Gel Preparation

Gels were prepared in a class II laminar flow hood using endotoxin free reagents and sterile, disposable or autoclaved consumables. All reagents and consumables were decontaminated with 70% v/v ethanol.

### Placebo Gels

Methylcellulose gel was prepared at 2%, 2.5% or 3% by slowly adding 0.4 g, 0.5 g or 0.6 g of methylcellulose (4000 cP at 2% solution; Sigma-Aldrich, UK; autoclaved to sterilise) to 10 ml of sterile deionised water at 80° C. in a glass bottle on a Stuart hotplate magnetic stirrer (Fisher scientific, UK). This solution was stirred for 15 minutes before being cooled to 37° C. while still being stirred. A further 10 ml of sterile deionised water was added and the solution was stirred for 30 minutes before being removed from the magnetic stirrer and stored at 4° C. overnight.

PF-127 gel was prepared at 20%, 25% and 30% by slowly adding 4 g, 5 g or 6 g of PF-127 (Sigma-Aldrich, UK) to 20 ml deionised water at 4° C. in a glass bottle on a magnetic stirrer. The solution was stirred at 4° C. for 40 minutes before being removed from the magnetic stirrer and stored at 4° C. overnight.

### Therapeutic Gels

Therapeutic methylcellulose gels were prepared in accordance with Example 2. Therapeutic PF-127 gel was prepared at 25% and 30% by slowly adding 3.75 g or 4.5 g of polymer powder to 15 ml composition at 4° C. in a glass bottle on a magnetic stirrer. The solution was stirred at 4° C. for 1 hour before being removed from the magnetic stirrer and stored at 4° C. overnight.

### Rheological Measurement

Rheological measurements were carried out to determine the sol-gel transition temperature of the placebo gel and therapeutic gel formulations.

### Photographic Observations

The consistency of the gels was also observed photographically. Gels were stored in 2 ml syringes at 4° C., 22° C. or 37° C. for 30 minutes before being injected onto an absorbent surface (Wypall blue roll; Fisher Scientific, UK). Photographs were taken immediately using a Canon EOS camera. Water was used as a negative control to indicate a liquid, and Nurofen® ibuprofen gel (Reckitt Benckiser, UK) was used as a positive control to indicate a gel consistency that is suitable for topical application.

### Fibroblast Proliferation Assay

Methylcellulose therapeutic gel and PF-127 therapeutic gel were prepared at 2.5% and 25% respectively. Fibroblast cells (MRC 5CV1) were cultured and harvested before being seeded into 96 well plates for alamarBlue® assay. FBS (2%) was used as the control, composition (2%) as the positive control, and Cisplatin at 15 μM as the negative control. Gel treatments were added to serum free media at 2%. Treatments were added to individual wells to ensure even dispersion of gel in each well.

### Optimisation of Composition Concentration

To determine the optimum concentration of composition in methylcellulose and PF-127 gels, the gels were prepared with varying concentrations of composition. Methylcellulose powder must be added to a liquid at 80° C. to ensure even dispersion so it was not possible to prepare methylcellulose gels with 100% composition due to protein denaturation at high temperatures. Therefore, methylcellulose gel was prepared with 50, 37.5, 25, 12.5, and 0% composition, while PF-127 gel was prepared with 100, 50, 37.5, 25, 12.5, and 0% composition. An alamarBlue® assay was performed. FBS (2%) was used as the control, composition (2%) as the positive control, and Cisplatin at 15 μM as the negative control. Gel treatments were added to serum free media at 2%.

### Gel Storage Solutions

To determine the length of time that therapeutic gels can be stored at room temperature and 4° C., methylcellulose therapeutic gel and PF-127 therapeutic gel were prepared at 2.5% and 25% respectively. The gels were stored at room temperature or 4° C., and some of the composition that was used to make the gels was aliquoted and stored at −80° C. until analysis. At 0 days, 7 days, 14 days, 28 days, 2 months, 3 months, and 6 months, alamarBlue® assays were performed. FBS (2%) was used as the control, composition (2%) as the positive control, and Cisplatin at 15 μM as the negative control. Gel treatments were added to serum free media at 2%.

### Results

### Rheological Measurements and Photographs of Placebo Gels

To measure the sol-gel transition temperature of methylcellulose and PF-127 placebos, an oscillation temperature ramp was performed on an AR-G2 rheometer. A solution has a storage modulus G′ (measure of elasticity) lower than the loss modulus G″ (measure of viscosity) so is more viscous than it is elastic. In contrast, a gel has a storage modulus G′ higher than the loss modulus G″ so is more elastic than it is viscous. The point at which G′ is equal to G″ is known as the sol-gel transition temperature. The phase angle δ is another indication of the gelling point. In solutions, the lowest frequency has the highest phase angle δ and the highest frequency has the lowest phase angle δ. The point at which the frequencies cross over is the sol-gel transition temperature.

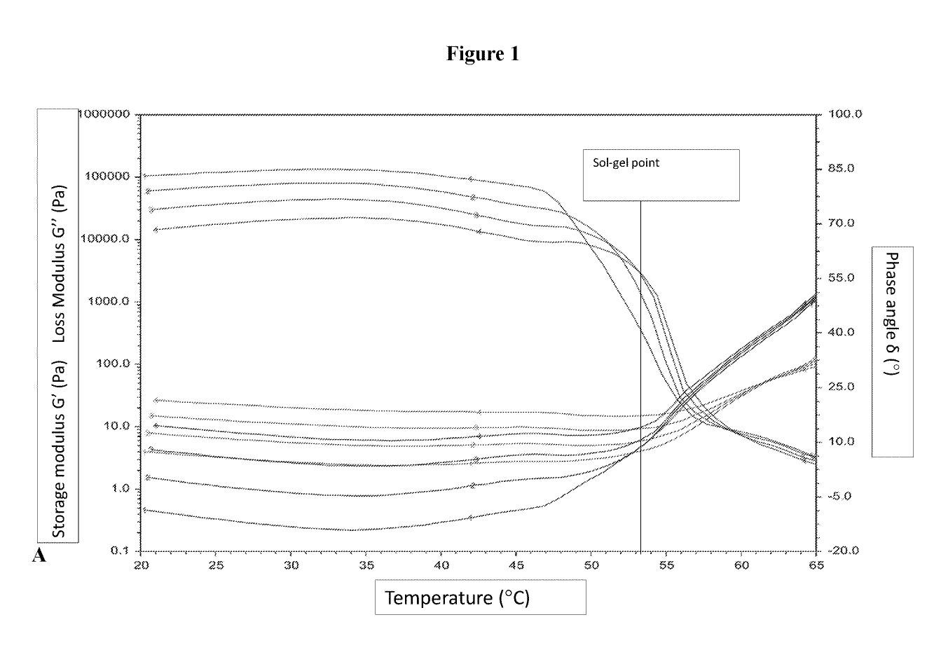

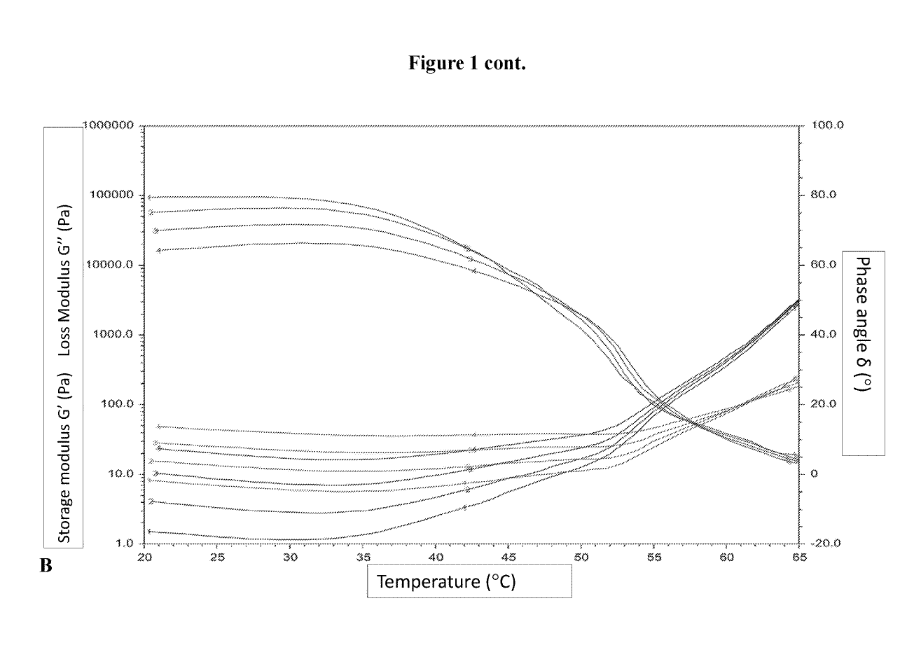

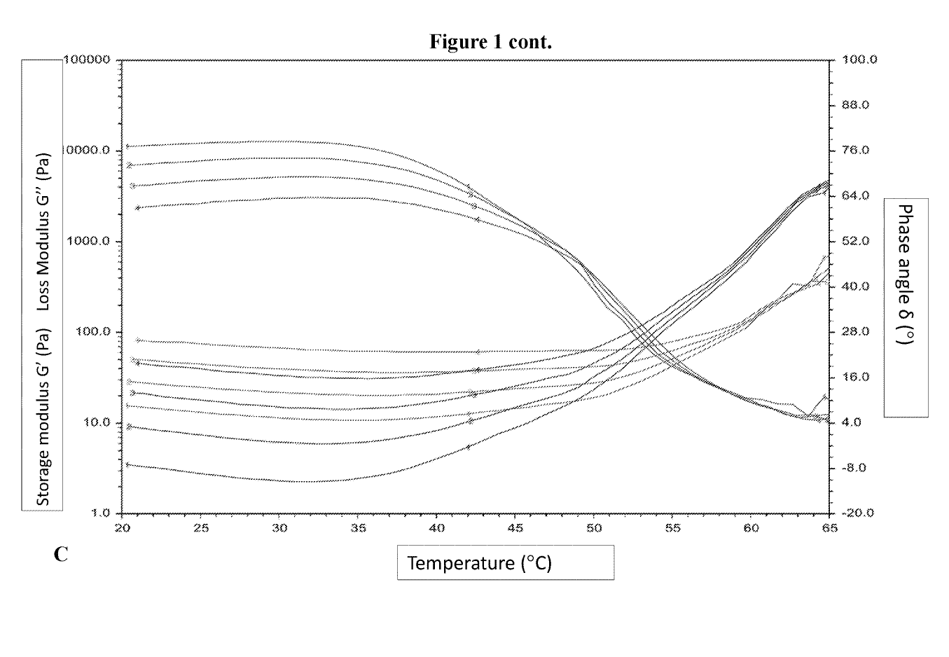

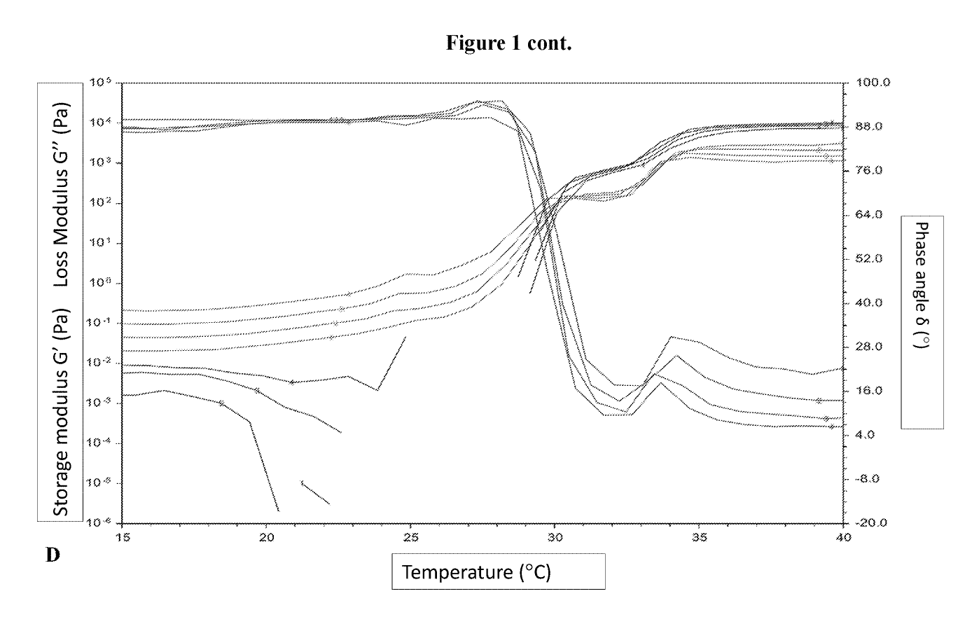

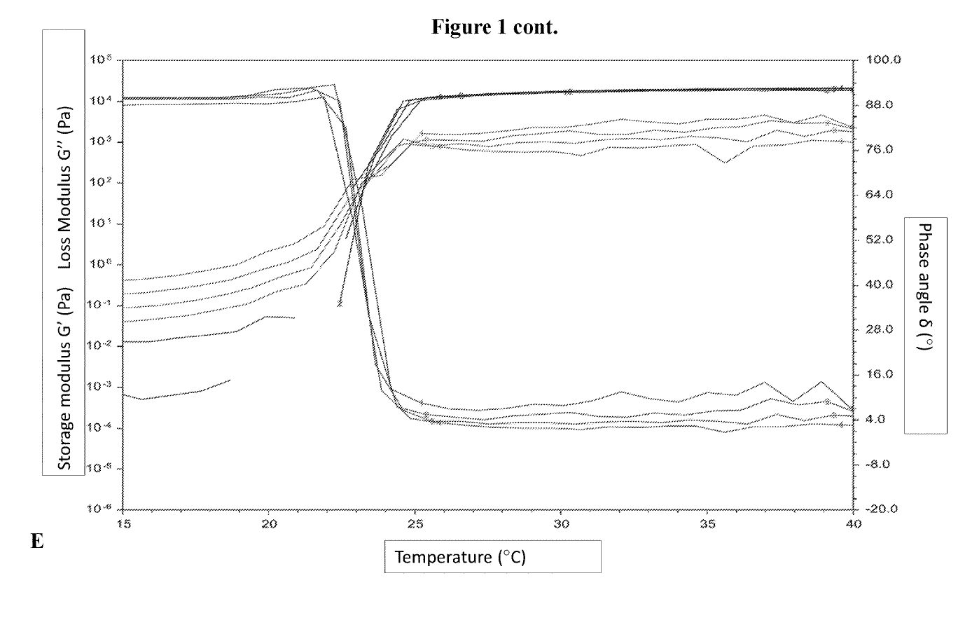

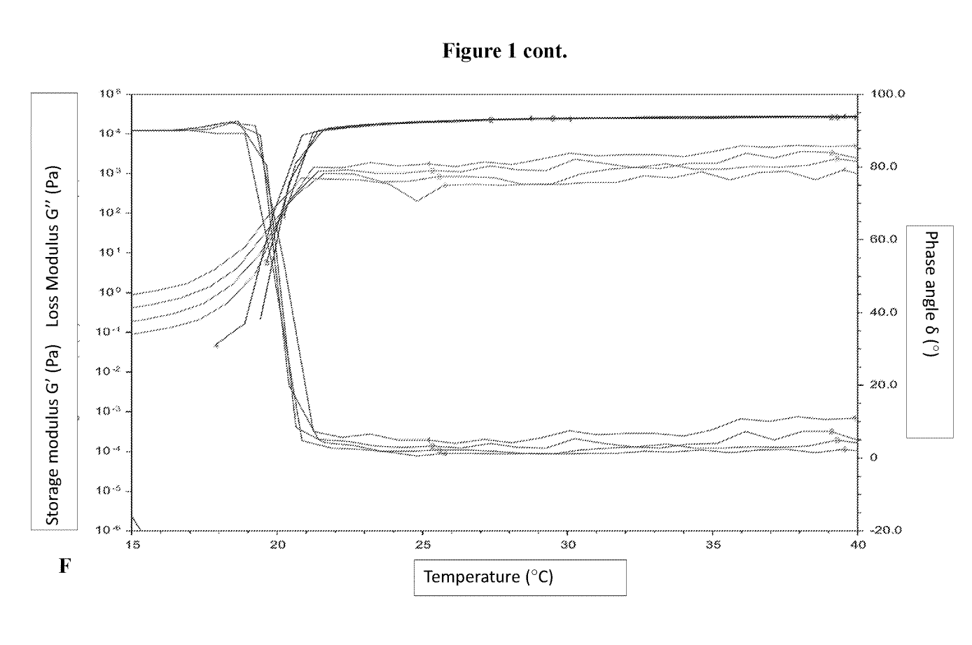

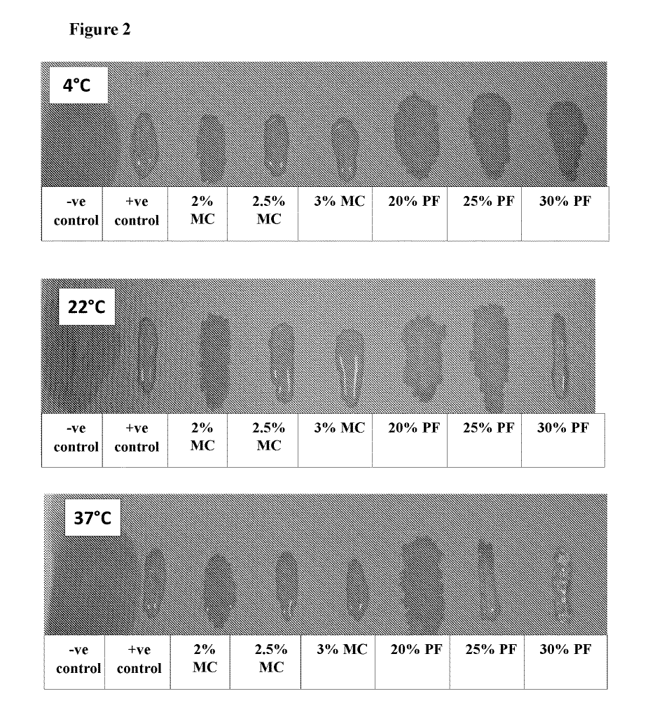

At room temperature (22° C.) and body temperature (37° C.), methylcellulose placebos (2.0-3.0%) were found to be more viscous than elastic (FIGS. 1 A-C) so were technically viscous liquids even though they appeared visually to be gels (FIG. 2). PF-127 placebos (20-30%) were found to be gels at body temperature but their status at room temperature was dependent upon the polymer concentration (FIGS. 1 D-F). Increasing the polymer concentration decreased the sol-gel transition temperature for both methylcellulose and PF-127 placebos (Table 3).

TABLE 3
Sol-gel transition temperatures of methyl
cellulose and PF-127 placebos

| Polymer | Concentration | Sol-gel point |
|---|---|---|
| Methyl cellulose | 2.0% | 53-55° C. |
| | 2.5% | 50-51° C. |
| | 3.0% | 49-50° C. |
| PF-127 | 20% | 29-31° C. |
| | 25% | 23-24° C. |
| | 30% | 20-21° C. |

Photographs were used to provide visual observations of gel viscosity at 4° C., 22° C. and 37° C. (FIG. 2). Methylcellulose did not appear to change in viscosity between 4° C., and 37° C. This is consistent with the rheological measurements which indicate that there is no increase in viscosity until above 50° C. PF-127 can be clearly seen to increase in viscosity with increasing temperature which is also consistent with rheological data. Following these observations, 2.5% was selected as the optimum concentration for methylcellulose while 25% and 30% were selected as optimum concentrations for PF-127.

### Rheological Measurements and Photographs of Therapeutic Gels

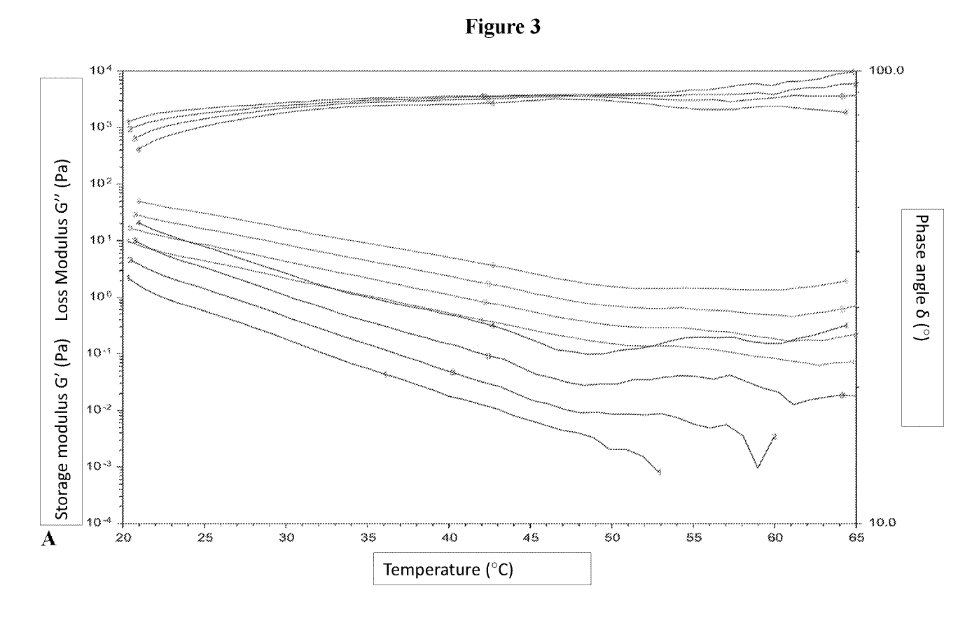

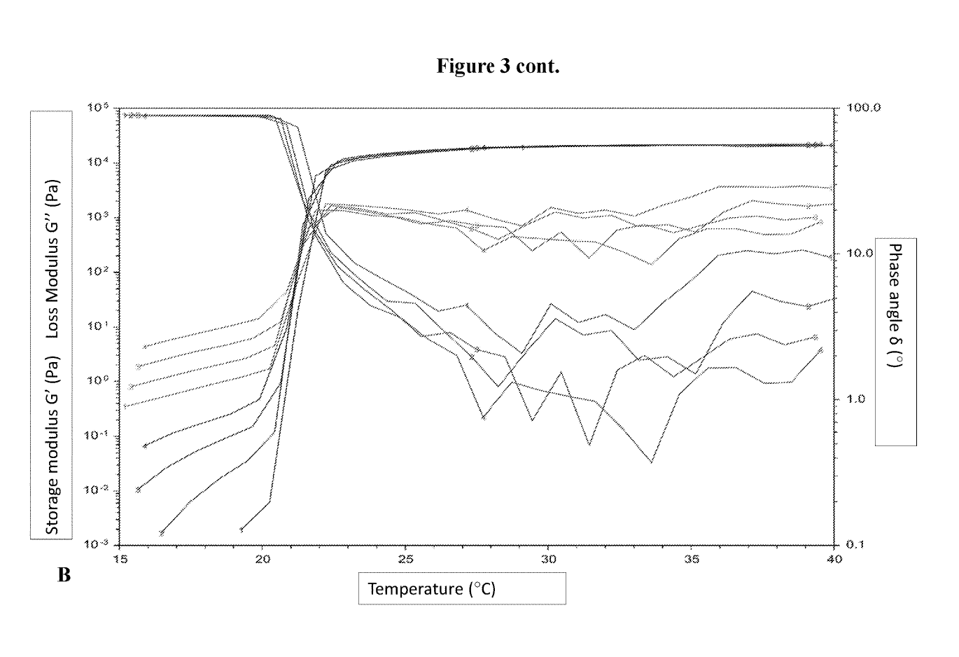

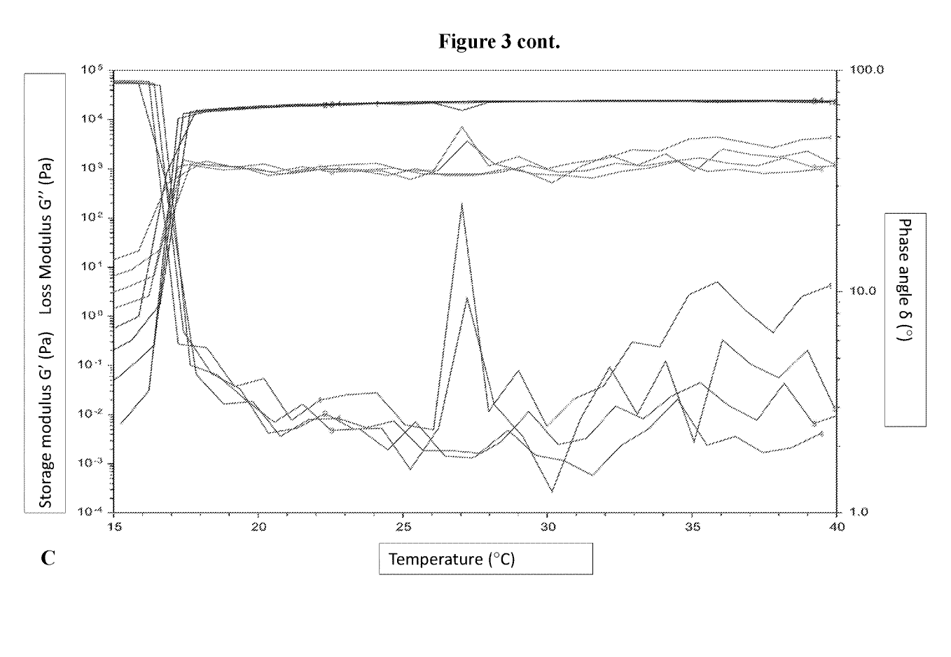

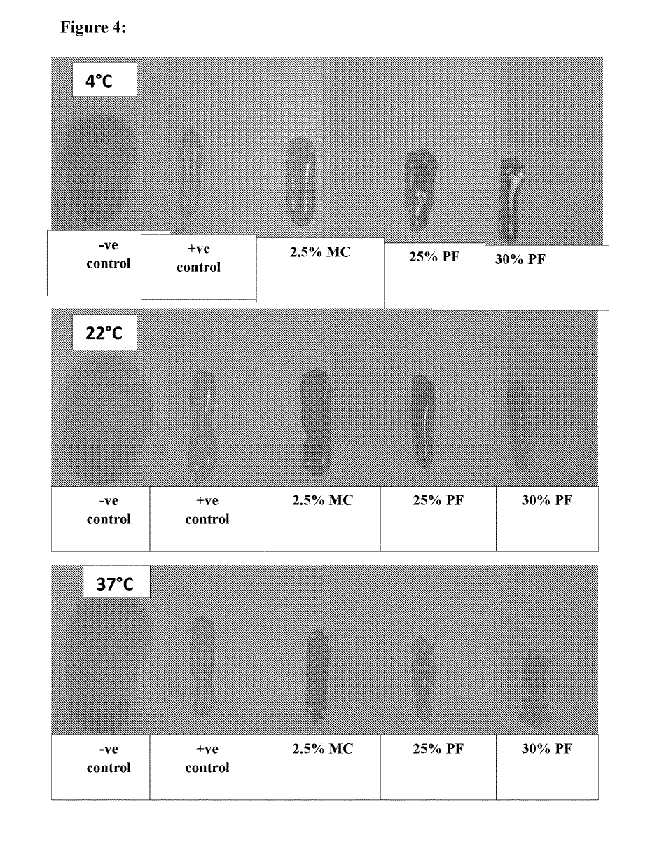

To observe the effect of composition on methylcellulose and PF-127, the oscillation temperature ramp and photographs were repeated for gels prepared with composition. Addition of composition to the gels had an effect on the sol-gel transition temperature (Table 4). Methylcellulose prepared with composition did not form a gel between 20-65° C. (FIG. 3A). However, at 37° C. the viscosity of methylcellulose with composition is still similar to that of the positive control (FIG. 4). In contrast, composition was found to improve the ability of PF-127 to form a gel, lowering the sol-gel transition temperature (FIG. 3B-C). The increase in viscosity can also be observed visually in FIG. 4. PF-127 PL (30%) was found to be very thick and consequently difficult to inject. Therefore, 2.5% methylcellulose and 25% PF-127 were selected as the optimum concentrations for composition gel formation.

TABLE 4
Sol-gel transition temperatures of methylcellulose
and PF-127 placebo and PL containing gels.

| | | Sol-gel point | Sol-gel point |
|---|---|---|---|
| Polymer | Concentration | (placebo) | (with PL) |
| Methylcellulose | 2.5% | 50-51° C. | N/A |
| PF-127 | 25% | 23-24° C. | 21-22° C. |
| | 30% | 20-21° C. | 16-17° C. |

### Optimisation of Composition Concentration

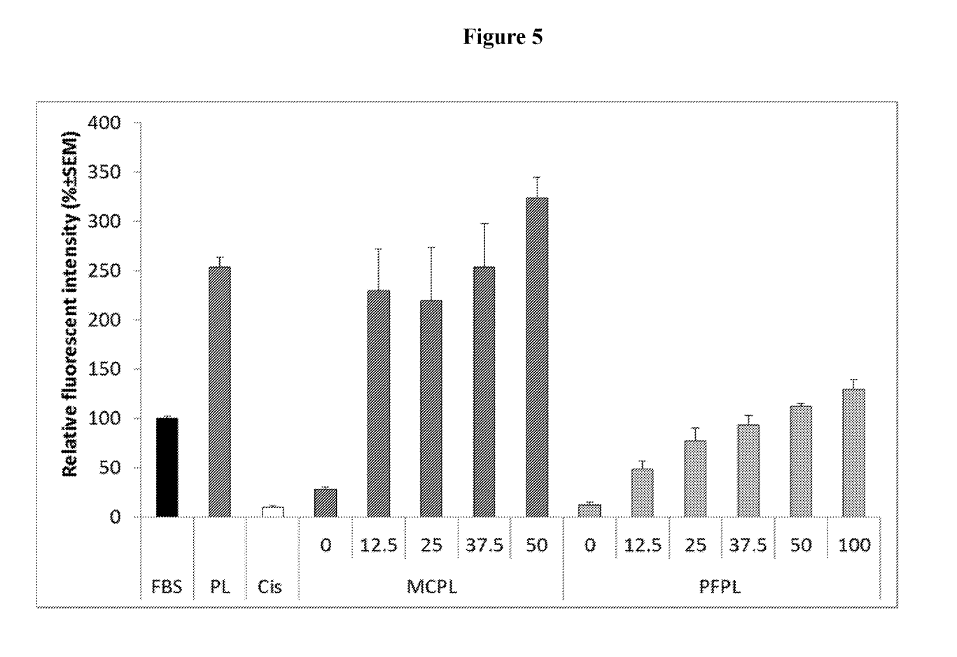

To investigate the proliferative capacity of therapeutic gels on fibroblast cells, fibroblasts were cultured for 48 hours with therapeutic gels containing various concentrations of composition. The results, shown in FIG. 5, indicate that methylcellulose therapeutic gel promoted significantly more fibroblast proliferation than the FBS control. There was no significant difference between PF-127 therapeutic gels and the FBS control. There was a general trend towards increased cell numbers as the concentration of composition increased but this was not significant. The top concentrations of composition were selected for further experiments (50% for methylcellulose and 100% for PF-127).

### Storage of Therapeutic Gel

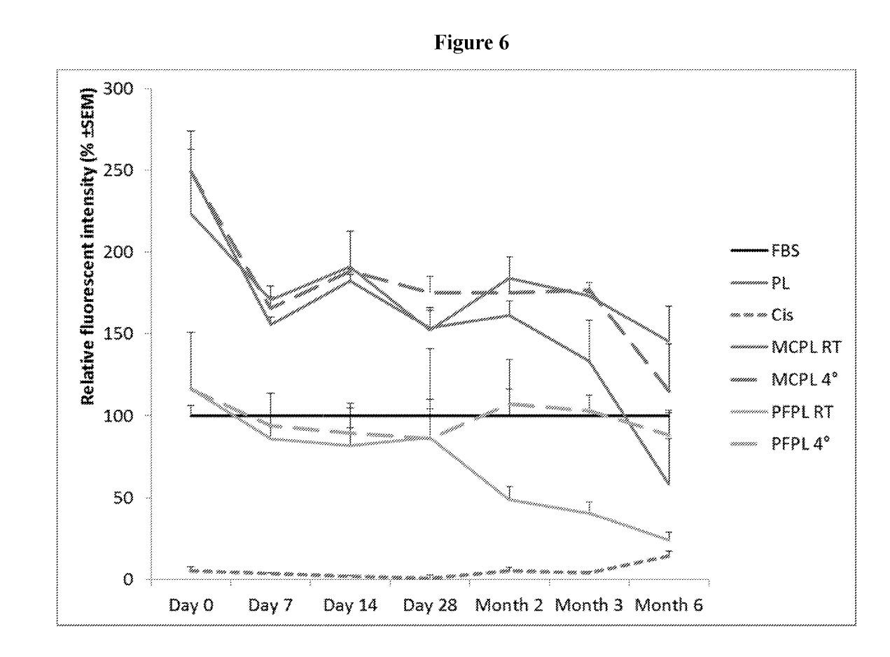

To determine the length of time that therapeutic gels can be stored without losing their beneficial effects on fibroblast proliferation, the gels were stored for a period of 0 days, 7 days, 14 days, 28 days, 2 months, 3 months or 6 months at room temperature or 4° C. before being used to culture fibroblasts and compared to an FBS and composition control. The results are shown in FIG. 6. Methylcellulose therapeutic gel stored at 4° C. for up to 3 months caused significantly more fibroblast proliferation (P≦0.01) than FBS control. When this methylcellulose therapeutic gel was stored at room temperature, the proliferative effect was only significantly greater than FBS for up to 2 months (P≦0.01). PF-127 therapeutic gel stored at 4° C. did not have any significant difference in fibroblast proliferation compared with FBS. However, when PF-127 therapeutic gel was stored at room temperature for over 28 days it had a significantly worse proliferative effect on fibroblasts than FBS (P≦0.05). As would be expected, storage at 4° C. preserved the biological properties of the therapeutic gel for longer than storage at room temperature.

### Conclusions

Sol-gel transition temperature is a key parameter for defining gel products. Rheological measurements of both methylcellulose and PF-127 gels indicated a decrease in sol-gel transition temperature (G′=G″) with increasing polymer concentration, an observation which has been reported previously. At body temperature, methylcellulose (2.0-3.0%) was found to be a viscous solution while PF-127 (20%-30%) was a gel. Addition of composition to PF-127 was found to lower the sol-gel transition temperature by 2 to 5° C. Gelation of PF-127 can be attributed to increases in temperature causing formation of micelles followed by ordered packing of these micelles into a cubic structure as the temperature continues to increase. In contrast, addition of composition to methylcellulose was found to prevent gel formation. At low temperatures, methylcellulose is water soluble due to the formation of cage like structures which surround the hydrophobic methyl groups. Increasing the temperature disrupts these cage structures and exposes the hydrophobic groups, leading to the formation of hydrophobic aggregates, producing a gel. Addition of salts to methylcellulose has been reported to either assist or supress the sol-gel transition. Salts may compete with the polymer for water, disrupting some of the cage like structures and mimicking an increase in temperature. On the other hand, salts may sit between methylcellulose chains, repelling water from the chains and making it more difficult for hydrophobic aggregates to form. Composition might have had this latter effect on methylcellulose since sol-gel transition was not observed between 20-65° C.

Visual examination of the gels was used to select the gel with the desired viscosity for topical application. The viscosity of the gels could be seen to increase as the polymer concentration increased which is in keeping with rheological measurements. Following these tests, 2.5% methylcellulose and 25% PF-127 were chosen as the optimum concentration for preparing therapeutic gel. Since these polymers are comparable in price, the use of methylcellulose would reduce the cost of the polymer powder by a factor of ten so this is a more commercially appealing option than PF-127.

Methylcellulose and PF-127 gels containing the maximum concentration of composition (50% composition for Methylcellulose and 100% PL for PF-127) appeared to induce fibroblast proliferation to a greater extent than lower concentrations. Methylcellulose therapeutic gels (12.5-50% composition) were found to induce significantly more cell proliferation than the FBS control, and similar levels to the composition positive control. In contrast, PF-127 composition gels (12.5-100%) were not statistically different to the FBS control. This may be due to the higher viscosity of PF-127 at 37° C. which limited diffusion of growth factors out of the gel. The maximum composition concentrations were chosen as the optimum for future gel preparation.

Finally, the shelf life of therapeutic gels was evaluated at room temperature and 4° C. Methylcellulose therapeutic gel retained its proliferative capacity for up to 2 months at room temperature and up to 3 months at 4° C. PF-127 therapeutic gel had significantly worse proliferative effect on fibroblasts after 28 days at room temperature but was able to maintain its effects for up to 6 months when stored at 4° C. However, PF-127 therapeutic gel induced lower levels of proliferation than methylcellulose therapeutic gel at all time-points. In addition, PF-127 therapeutic gel was in solution at 4° C. causing separation of composition from the polymer. This led to a non-uniform product after storage for a short period of time.

In summary, this Example developed a therapeutic gel which was able to successfully promote fibroblast proliferation for up to 3 months after preparation. The optimum gel contained 50% composition and 2.5% methylcellulose as the carrier matrix. This matrix may improve the storage life of platelet gel and support sustained release of growth factors. PF-127 did not perform as well as methylcellulose which may be due to reduced diffusion of growth factors from this gel. The optimised gel can be stored at 4° C. for up to 3 months.

### Example 4—Screening of Growth Factors in Composition Using Luminex 7Plex Angiogenesis Panel

### Method

Compositions were prepared from 21 different platelet samples in accordance with Example 1. The resultant compositions were used to prepare therapeutic gels according to Example 2. Each final gel comprised 2.5% methylcellulose, 0.5x PBS and 50% composition.

The growth factor content of each therapeutic gel was analysed using the Luminex 7plex Angiogenesis Panel kit (part #893605; lot #311544). 100 μl therapeutic gel was dispensed in triplicate to dilution tubes, 400p diluent added, and the samples mixed carefully. 100 μl of each diluted sample was transferred to a 96-well assay plate. The excess of each of the 21 therapeutic gels was stored at −80° C. for future use (see Example 5).

The Standard Cocktail was reconstituted with diluent and used to make a standard curve as per the kit instructions. 100 μl of each standard was transferred to column 1 and 2 of the 96-well plate.

A microparticle suspension was prepared by suspending a cocktail of 50 μl of each microparticle in 5 ml microparticle diluent. The suspension was mixed thoroughly by vortexing, and 50 μl added to each well of the 96-well plate. The plate was sealed with plate sealer foil and incubated for 2 hours at room temperature on a horizontal shaker at 800 rpm.

50 μl of each biotin-conjugated antibody was added to the vial of biotin antibody diluent. Luminex wash buffer was prepared by adding 20 ml 25x Wash Buffer concentrate to 480 ml H₂O. The assay plate was washed three times in Wash Buffer, and 50 μl of diluted biotin-conjugated antibody added to each well. The plate was sealed with foil and incubated for 1 hour at room temperature on a horizontal shaker at 800 rpm. Meanwhile, the lasers of the Luminex machine were warmed up, and the machine calibrated. The analyte parameters were entered as shown in Table 5. The sample template for the machine was set up.

TABLE 5
analyte parameters.

| Analyte | Microparticle Region |
|---|---|
| angiopoietin | 25 |
| bFGF | 13 |
| PDGF-AA | 18 |
| PDGF-BB | 19 |
| Thrombospondin-2 | 21 |
| VEGF | 39 |
| VEGF-D | 22 |

Strepavidin-PE was made up in accordance with kit instructions. The plate was washed as before and streptavidin-PE to each well. The plate was covered with foil sealer and incubated for 30 minutes at room temperature on a horizontal shaker at 800 rpm. The plate was washed as before and samples resuspended in 100 μl wash buffer. Samples were then loaded to the Luminex machine.

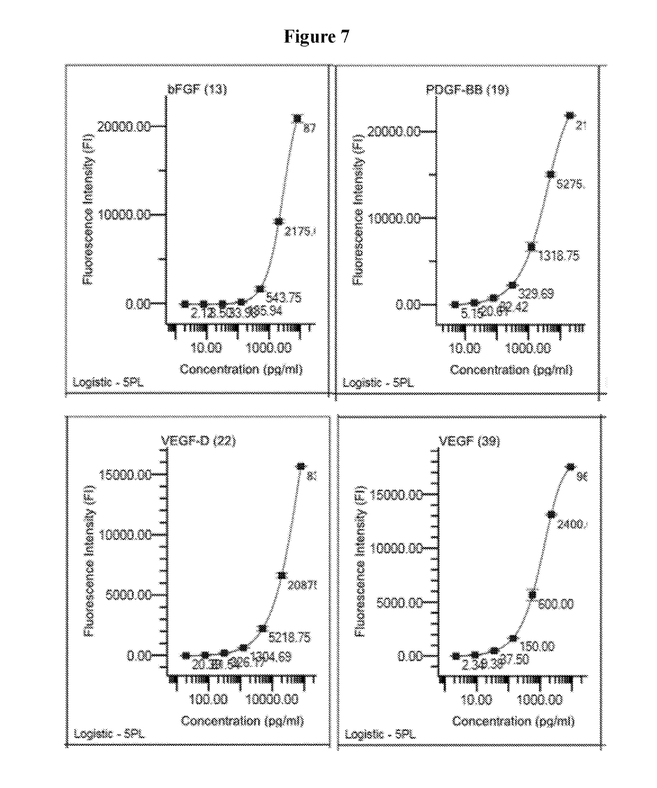

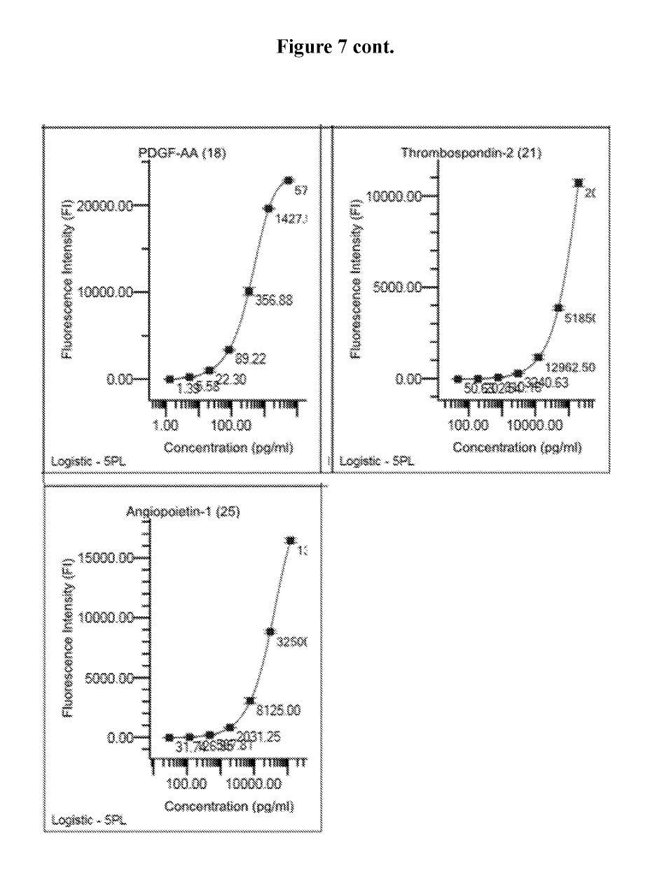

A standard curve was generated using five parameter logistic (5-PL) curve fit (FIG. 7).

### Results

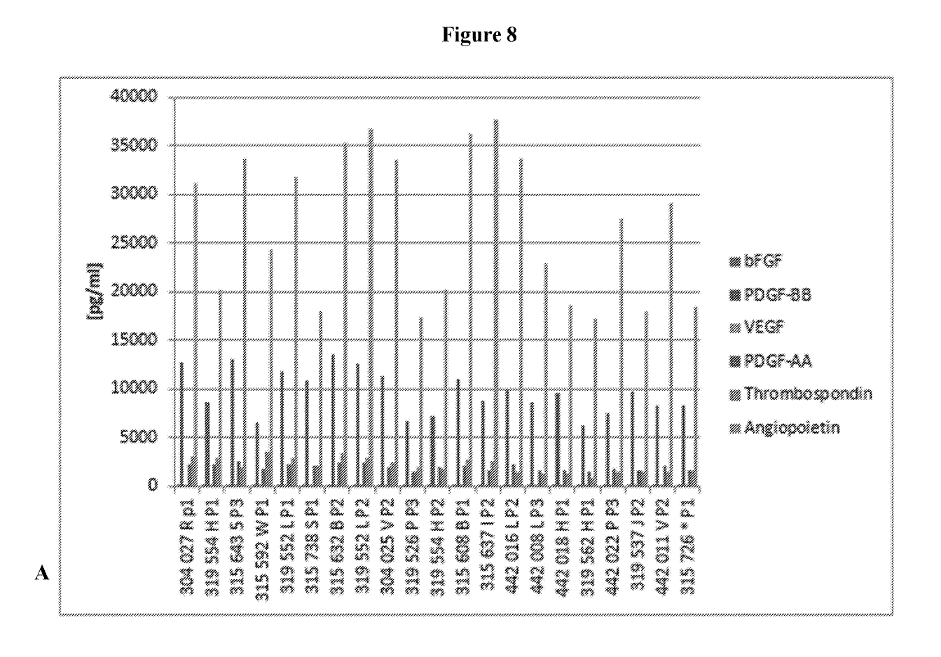

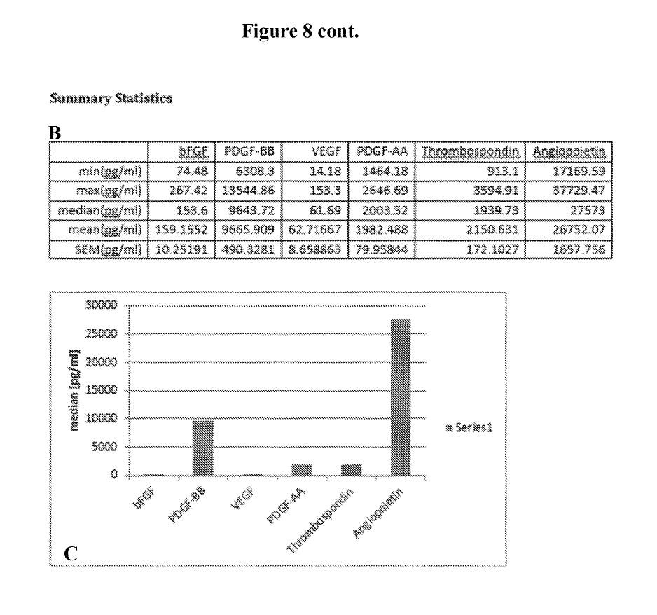

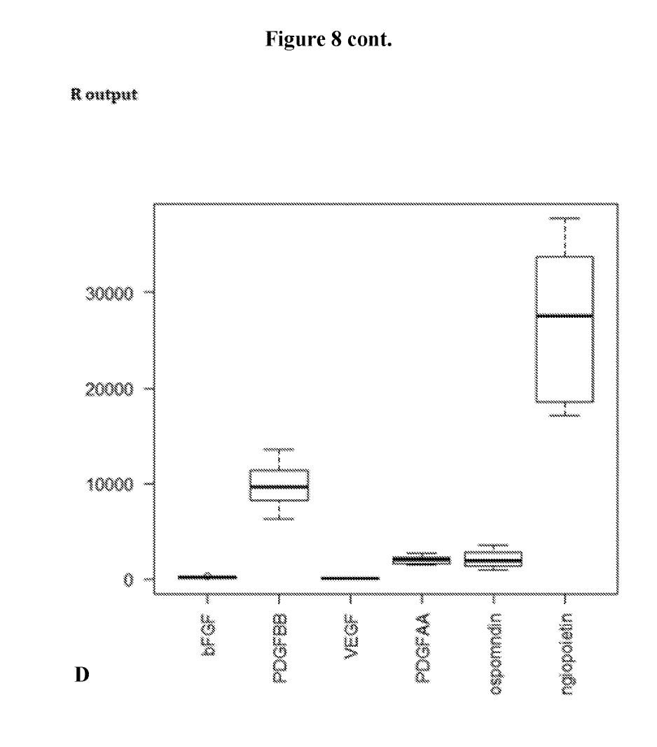

Results are shown in FIG. 8. FIG. 8A shows the concentration of bFGF, PDGF-BB, VEGF, PDGF-AA, thrombospondin and angiopoeitin in each therapeutic gel analysed. Levels of VEGF-D were below the detection limit and are not shown. FIG. 8B summarises the minimum, maximum, median, mean and SEM for each growth factor, calculated across the sample set. FIG. 8C depicts the median concentration of each growth factor, and FIG. 8D presents this information as a box and whisker plot.

### Conclusions

Of the growth factors assayed, angiopoietin was the most plentiful factor, followed by PDGF-BB. VEGF-D was not detected.

### Example 5—Effect of Storage on Growth Factor Concentration

### Method

The excess of each of the 21 therapeutic gels prepared in Example 4 was stored at 4° C. for 2 weeks. There was no obvious sign of stratification of protein in the tubes of gel. Each tube was mixed gently by rolling before used in this Example.

The growth factor content of each therapeutic gel was analysed using the Luminex 7plex Angiogenesis Panel kit (part #893605; lot #311544). 50 μl therapeutic gel was dispensed in triplicate to dilution tubes, 200 μl diluent added, and the samples mixed carefully. 100 μl of each diluted sample was transferred to a 96-well assay plate.

The Standard Cocktail was reconstituted with diluent and used to make a standard curve as per the kit instructions. 100 μl of each standard was transferred to column 1 and 2 of the 96-well plate.

A microparticle suspension was prepared by suspending a cocktail of 50 μl of each microparticle in 5 ml microparticle diluent. The suspension was mixed thoroughly by vortexing, and 50 μl added to each well of the 96-well plate. The plate was sealed with plate sealer foil and incubated for 2 hours at room temperature on a horizontal shaker at 800 rpm.

50 μl of each biotin-conjugated antibody was added to the vial of biotin antibody diluent. Luminex wash buffer was prepared by adding 20 ml 25x Wash Buffer concentrate to 480 ml H₂O. The assay plate was washed three times in Wash Buffer, and 50 μl of diluted biotin-conjugated antibody added to each well. The plate was sealed with foil and incubated for 1 hour at room temperature on a horizontal shaker at 800 rpm. Meanwhile, the lasers of the Luminex machine were warmed up, and the machine calibrated. The analyte parameters were entered as shown in Table 6. The sample template for the machine was set up.

TABLE 6
analyte parameters.

| Analyte | Microparticle Region |
|---|---|
| angiopoietin | 25 |
| bFGF | 13 |
| PDGF-AA | 18 |
| PDGF-BB | 19 |
| Thrombospondin-2 | 21 |
| VEGF | 39 |
| VEGF-D | 22 |

Strepavidin-PE was made up in accordance with kit instructions. The plate was washed as before and streptavidin-PE to each well. The plate was covered with foil sealer and incubated for 30 minutes at room temperature on a horizontal shaker at 800 rpm. The plate was washed as before and samples resuspended in 100 μl wash buffer. Samples were then loaded to the Luminex machine.

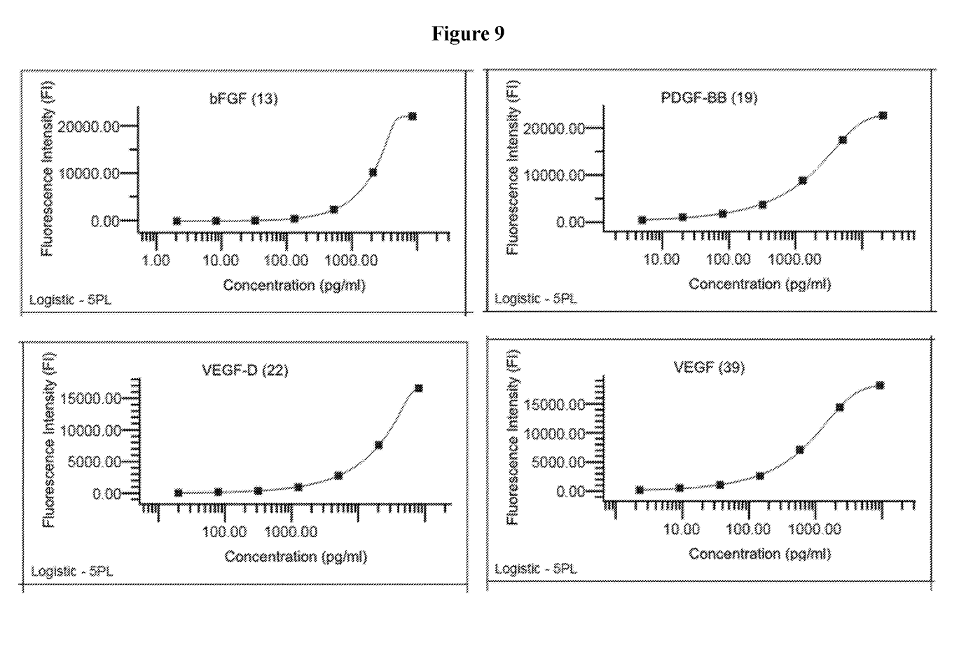

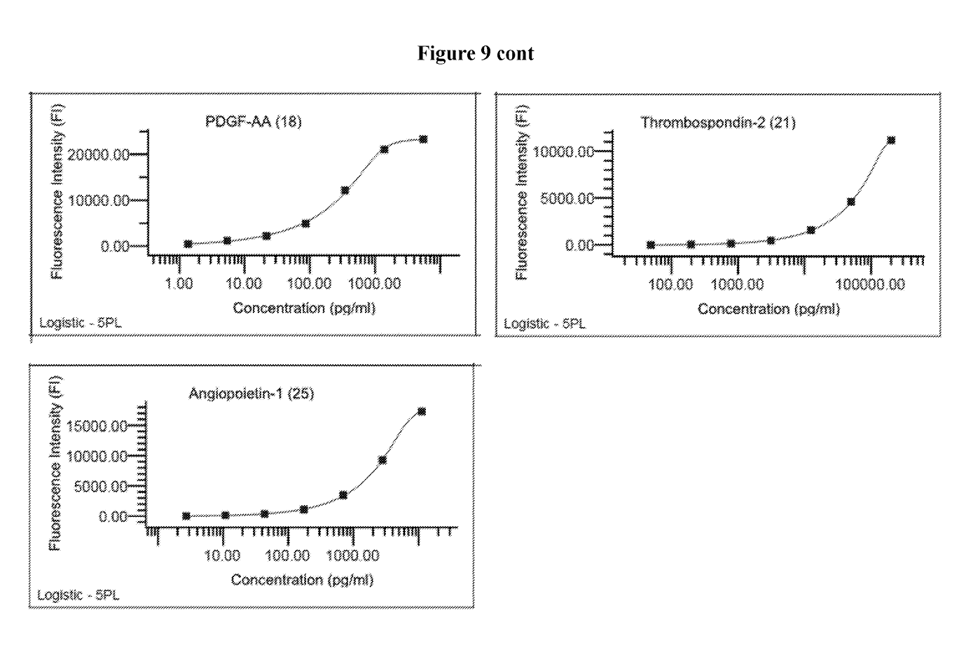

A standard curve was generated using five parameter logistic (5-PL) curve fit (FIG. 9).

### Results

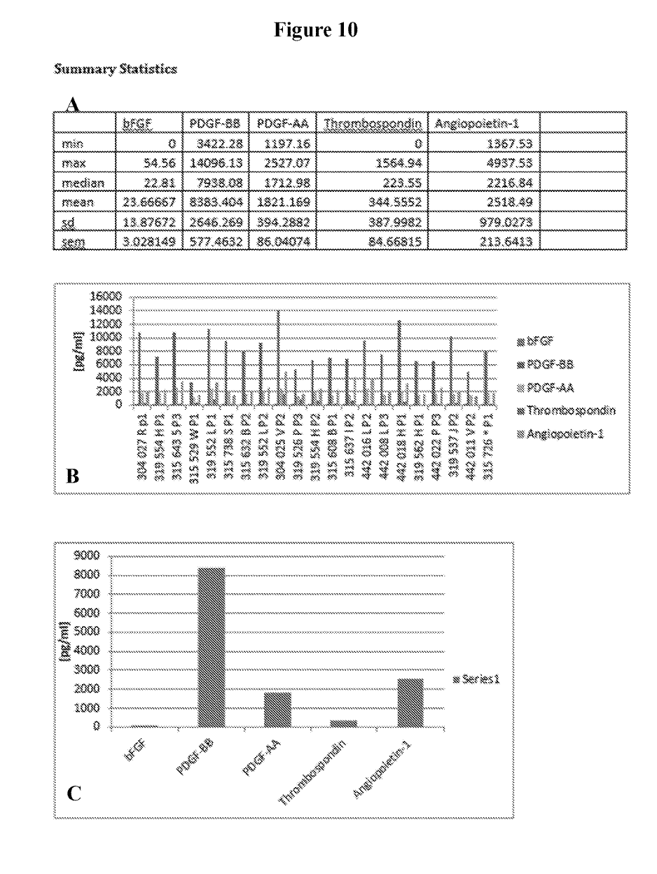

Results are shown in FIG. 10. FIG. 10B shows the concentration of bFGF, PDGF-BB, PDGF-AA, thrombospondin and angiopoeitin in each therapeutic gel analysed. FIG. 10A summarises the minimum, maximum, median, mean, SD and SEM for each growth factor, calculated across the sample set. FIG. 10C depicts the median concentration of each growth factor. VEGF and VEGF-D were undetectable.

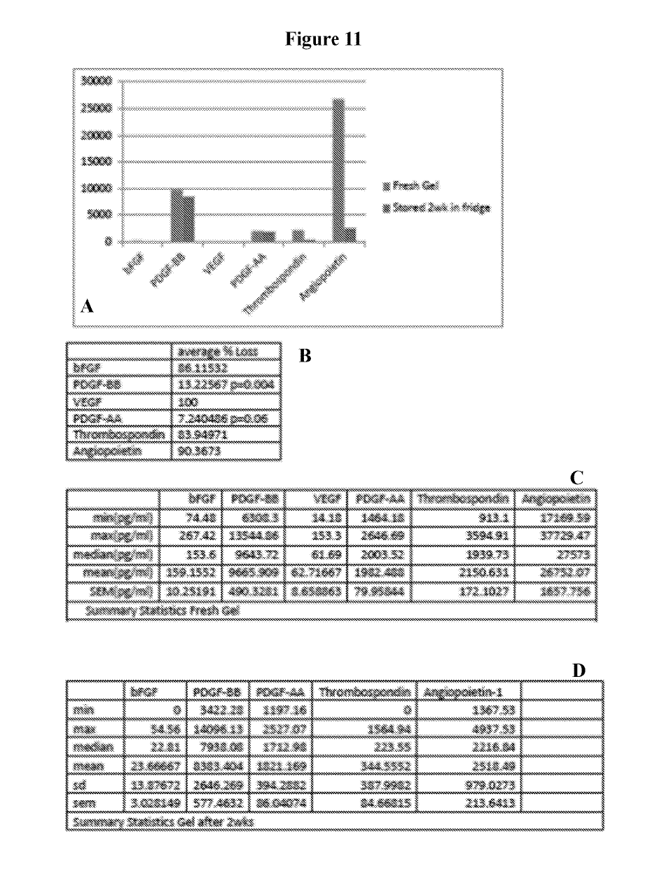

FIG. 11 compares the growth factor content of fresh and 2-week stored gel. FIG. 11A depicts the median growth factor concentration of both fresh and 2-week stored gel. FIG. 11B shows the average percentage loss of growth factor from fresh therapeutic gel after 2 weeks of storage.

### Conclusions

After 2 weeks of storage, PDGF-BB was the most plentiful growth factor in the therapeutic gels, followed by angiopoietin. However, all of the growth factors except PDGF-BB and PDGF-AA lost more than 80% of their activity after 2 weeks of storage.

1. A composition comprising at least one basic fibroblast growth factor (bFGF) isoform and at least one platelet-derived growth factor (PDGF) isoform.

2. A composition according to claim 1, wherein the at least one bFGF isoform is monomeric or dimeric.

3. A composition according to claim 1, wherein the bFGF isoform is homodimeric.

4. A composition according to claim 1, wherein the bFGF isoform is heterodimeric.

5. A composition according to claim 1, wherein the at least one bFGF isoform is one of 18 kDa bFGF (SEQ ID NO: 1) or a variant thereof, 22 kDa bFGF (SEQ ID NO: 2) or a variant thereof, 22.5 kDa bFGF or a variant thereof, 24 kDa bFGF (SEQ ID NO: 3) or a variant thereof and 34 Da bFGF (SEQ ID NO: 4) or a variant thereof.

6. A composition according to claim 1, wherein the at least one PDGF isoform is homodimeric.

7. A composition according to claim 6, wherein the at least one PDGF isoform comprises two PDGF-A subunits (SEQ ID NO: 5) or a variant thereof, two PDGF B subunits (SEQ ID NO: 6) or a variant thereof, two PDGF-C subunits (SEQ ID NO: 7) or a variant thereof, or two PDGF-D subunits (SEQ ID NO: 8) or a variant thereof.

8. A composition according to claim 1, wherein the at least one PDGF isoform is heterodimeric.

9. A composition according to claim 8, wherein the at least one PDGF isoform comprises one PDGF-A subunit (SEQ ID NO: 5) or a variant thereof and one PDGF-B subunit (SEQ ID NO: 6) or a variant thereof.

10. A composition according to claim 1, wherein the bFGF is recombinant bFGF and/or the PDGF isoform is a recombinant PDGF isoform.

11. A composition according to claim 1, wherein the composition does not comprise detectable levels of placental growth factor (PlGF).

12. A composition according to claim 1, wherein the composition does not comprise detectable levels of at least one of activin A, activin C, activin β, artemin, brain-derived neurotrophic factor (BDNF), bone morphogenetic protein 15 (BMP-15), BMP-2, BMP3, BMP-3b, BMP-4, BMP-5, BMP-6, BMP-7, BMP-8, beta nerve growth factor (b-NGF), betacellulin (BTC), ciliary neurotrophic factor (CNTF), connective tissue growth factor (CTGF), Dickkopf-related protein 1 (Dkk-1), endocan, eotaxin, epiregulin, fibroblast growth factor 10 (FGF-10), FGF-11, FGF-12, FGF-13, FGF-16, FGF-17, FGF-18, FGF-19, FGF-20, FGF-21, FGF-23, FGF-4, FGF-6, FGF-8, FGF-9, granulocyte colony-stimulating factor (GCSF), growth differentiation factor 1 (GDF-1), GDF-11, GDF-15, GDF-3, GDF-5, GDF-8, GDF-9, glial cell-derived neurotrophic factor (GDNF), growth hormone 1 (GH-1), granulocyte macrophage colony-stimulating factor (GM-CSF), chemokine (C-X-C motif) ligand 1 (CXCL1), CXCL2, CXCL3, Heparin-binding EGF-like growth factor (HB-EGF), hepatocyte growth factor (HGF), interferon γ (IFNγ), insulin-like growth factor 1 (IGF-1), IGF-11, interleukin 10 (IL-10), IL-12, IL-15, IL-1a, IL-1b, IL-1RA, IL-113, IL-2, IL-2R, IL-5, IL-6, IL-7, inhibin A, interferon gamma-induced protein 10 (IP-10), lefty A, leukaemia inhibitory factor (LIF), monocyte chemotactic protein 1 (MCP-1), macrophage colony-stimulating factor (M-CSF), milk fat globule-EGF factor 8 protein (MFG-E8), MIG, macrophage inflammatory proteins (MIP-1a), MIP-1b, neurturin, nephroblastoma overexpressed gene (NOV), neurotrophin 3 (NT-3), NT-4, platelet-derived growth factor C (PDGF-C), persephin, progranulin, soluble CD40 ligand (sCD40L), Skp, Cullin, F-box containing complex (SCF) and soluble vascular cell adhesion molecule 1 (sVCAM-1).

13. A composition according to claim 1, wherein the composition comprises detectable levels of at least one of angiopoietin, CXCL4, CXCL7, CXCL12, epidermal growth factor (EGF), interleukin 4 (IL-4), IL-8, IL-13, IL-17, interferon α (IFNα), lipoxin A4 (LXA4), protease-activated receptor 4 (PAR4), chemokine (C-C motif ligand) 5 (CCL5/RANTES), platelet-derived growth factor AA (PDGF-AA), PDGF-AB, transforming growth factor beta 1 (TGF-β1), TGF-β2, thrombospondin, tumour necrosis factor alpha (TNFα) and vascular endothelial growth factor D (VEGF-D).

14. A composition according to claim 1, wherein the composition comprises detectable levels of at least one of angiopoietin, CXCL4, CXCL7, CXCL12, lipoxin A4 (LXA4), protease-activated receptor 4 (PAR4) and thrombospondin.

15. A composition according to claim 1, wherein the composition comprises a detectable level of angiopoietin.

16. A composition according to claim 1, wherein the composition comprises detectable levels of at least one bFGF isoform, PDGF-AB and PDGF-BB.

17. A composition according to claim 16, wherein the composition comprises any or all of EGF, TGF-β1, TGF-β2, IGF and VEGF.

18. A composition according to claim 1, wherein the composition comprises any or all of transforming growth factor alpha (TGF-α), acidic fibroblast growth factor (aFGF), TGF-β3 and FGF-7.

19. A composition according to claim 1, wherein the concentration of bFGF is within the range of from about 50 pg to about 500 pg per millilitre of composition.

20. A composition according to claim 1, wherein the total concentration of PDGF is from about 5000 pg to about 25000 pg per millilitre of composition.

21. A composition according to claim 1, wherein the ratio of bFGF to total PDGF is from about 1:10 to about 1:500.

22. A composition according to claim 1, wherein the composition further comprises a pharmaceutically acceptable polymer.

23. A composition according to claim 1, wherein the composition comprises a gel.

24. A composition according to claim 22, wherein the polymer is an alginate polymer, a double ester polymer of ethylidene, poly(D,L-lactide-co-glycolide) (PLGA), poly(vinyl alcohol) (PVA), polyperfluorooctyloxycaronyl-poly(lactic acid) (PLA-PFO) or another block copolymer.

25. A composition according to claim 22, wherein the polymer is a cellulose polymer.

26. A composition according to claim 25, wherein the cellulose polymer is carboxymethylcellulose, hydroxypropylmethylcellulose or methycellulose.

27. A composition according to claim 25, wherein the cellulose polymer concentration is 1.5% (w/w) to 4.0% (w/w) and the polymer has a molecular weight of 450,000 to 4,000,000.

28. A composition according to claim 22, wherein the composition has a viscosity in the range of 1000 to 500,000 mPa·s (cps) at room temperature.

29. A composition according to claim 28, wherein the viscosity is in the range of 50,000 to 150,000 mPa·s (cps) at room temperature.

30. A composition according to claim 1, further comprising a preservative selected from the group consisting of methylparaben, propylparaben and m-cresol.

31. A composition according to claim 1, wherein the composition has an osmolality in the range of 280 to 320 mOsmol/kg.

32. A composition according to claim 1, wherein the composition is xeno-free.

33. A composition according to claim 1, wherein the composition comprises one or more therapeutic cells.

34. A composition according to claim 33, wherein the one or more therapeutic cells comprise one or more progenitor cells of mesodermal lineage (PMLs), one or more immuno-modulatory progenitor (IMP) cells, one or more mesenchymal stem cells, one or more dendritic cells, one or more platelets, one or more fibroblasts, one or more myofibroblasts or a combination thereof.

35. A composition according to claim 34, wherein the one or more PMLs (a) express detectable levels of CD29, CD44, CD73, CD90, CD105 and CD271 and (b) do not express detectable levels of CD14, CD34 and CD45.

36. A composition according to claim 35, wherein the one or more PMLs express detectable levels of CD62P (P-selectin) and/or CD62E (E-selectin).

37. A composition according to claim 34, wherein the one or more IMP cells express detectable levels of MIC A/B, CD304 (Neuropilin 1), CD178 (FAS ligand), CD289 (Toll-like receptor 9), CD363 (Sphingosine-1-phosphate receptor 1), CD99, CD181 (C-X-C chemokine receptor type 1; CXCR1), epidermal growth factor receptor (EGF-R), CXCR2 and CD126.

38. A composition according to claim 34, wherein the one or more IMP cells express detectable levels of one or more of CD10, CD111, CD267, CD47, CD273, CD51/CD61, CD49f, CD49d, CD146, CD55, CD340, CD91, Notch2, CD175s, CD82, CD49b, CD95, CD63, CD245, CD58, CD108, B2-microglobulin, CD155, CD298, CD44, CD49c, CD105, CD166, CD230, HLA-ABC, CD13, CD29, CD49e, CD59, CD73, CD81, CD90, CD98, CD147, CD151 and CD276.

39. A composition according to claim 33, wherein the one or more IMP cells express:
(a) detectable levels of one or more of CD156b, CD61, CD202b, CD130, CD148, CD288, CD337, SSEA-4, CD349, CD140b, CD10, CD111, CD267, CD47, CD273, CD51/CD61, CD49f, CD49d, CD146, CD55, CD340, CD91, Notch2, CD175s, CD82, CD49b, CD95, CD63, CD245, CD58, CD108, B2-microglobulin, CD155, CD298, CD44, CD49c, CD105, CD166, CD230, HLA-ABC, CD13, CD29, CD49e, CD59, CD73, CD81, CD90, CD98, CD147, CD151 and CD276; and/or
(b) detectable levels of one or more of CD72, CD133, CD192, CD207, CD144, CD41b, FMC7, CD75, CD3e, CD37, CD158a, CD172b, CD282, CD100, CD94, CD39, CD66b, CD158b, CD40, CD35, CD15, PAC-1, CLIP, CD48, CD278, CD5, CD103, CD209, CD3, CD197, HLA-DM, CD20, CD74, CD87, CD129, CDw329, CD57, CD163, TPBG, CD206, CD243 (BD), CD19, CD8, CD52, CD184, CD107b, CD138, CD7, CD50, HLA-DR, CD158e2, CD64, DCIR, CD45, CLA, CD38, CD45RB, CD34, CD101, CD2, CD41a, CD69, CD136, CD62P, TCR alpha beta, CD16b, CD1a, ITGB7, CD154, CD70, CDw218a, CD137, CD43, CD27, CD62L, CD30, CD36, CD150, CD66, CD212, CD177, CD142, CD167, CD352, CD42a, CD336, CD244, CD23, CD45RO, CD229, CD200, CD22, CDH6, CD28, CD18, CD21, CD335, CD131, CD32, CD157, CD165, CD107a, CD1b, CD332, CD180, CD65 and CD24.

40. A method of repairing a damaged tissue in a patient, comprising administering to the patient a therapeutically effective amount of the composition of any one of the preceding claims, and thereby treating the damaged tissue in the patient.

41. A method according to claim 40, wherein the tissue is damaged by injury or disease.

42. A method of inhibiting senescence in a patient, comprising administering to the patient a therapeutically effective amount of the composition of claim 1, and thereby treating senescent tissue in the patient.

43. A method according to claim 40, wherein the tissue is derived from mesoderm.

44. A method according to claim 43, wherein the tissue is tendon, ligament, cardiac, bone, cartilage, liver, kidney, lung tissue or vaginal tissue.

45. A method according to claim 44, wherein the method is for treating tendon, ligament, cardiac, bone, cartilage, liver, kidney, lung tissue or vaginal tissue injury or disease in the patient.

46. A method according to claim 44, wherein the method is for inhibiting tendon, ligament, cardiac, bone, cartilage, liver, kidney, lung tissue or vaginal tissue senescence in the patient.

47. A method according to claim 45, wherein the method is for treating vaginal atrophy.

48. A method of promoting hair growth in a patient, comprising administering to the patient a therapeutically effective amount of the composition of claim 1.

49. A method according to claim 40, wherein administration of the composition improves tissue regeneration and/or reduces apoptosis.

50. A composition according to claim 1 for use in a method of repairing damaged tissue in a patient, the method comprising administering to the patient a therapeutically effective amount of the composition.

51. A composition according to claim 1 for use in a method of inhibiting senescence in a patient in need thereof, the method comprising administering to the patient a therapeutically effective amount of the composition.

52. A composition according to claim 1 for use in a method of promoting hair growth in a patient, the method comprising administering to the patient a therapeutically effective amount of the composition.

53. A method of producing a population of cells for use in a method of treating damaged tissue or inhibiting senescence, or promoting hair growth, the method comprising culturing mononuclear cells (MCs), PMLs and/or IMPs in the presence of the composition defined in claim 1, and allowing at least some of the MCs, PMLs and/or IMPs to proliferate.

54. A method according to claim 53, wherein the MCs are peripheral blood mononuclear cells (PBMCs).

55. A method according to claim 53, wherein the PMLs are as defined in claim 35.

56. A method according to claim 53, wherein the IMPs are as defined in claim 37.

57. A method according to claim 53, wherein the method further comprises isolating the proliferated cells.
